(19) United States
(12) Patent Application Publication
(10) Pub. No.: US 2023/0123617 A1
(43) Pub. Date: Apr. 20, 2023

(54) VENTRAL STRIATUM ACTIVITY

(71) Applicants: Ichilov Tech Ltd., Tel-Aviv (IL); Ramot at Tel-Aviv University Ltd., Tel-Aviv (IL); The Royal Institution For The Advancement Of Learning/McGill University, Montreal (CA)

(72) Inventors: Talma HENDLER, Tel-Aviv (IL); Neomi SINGER, Givatayim (IL); Robert ZATORRE, Montreal (CA); Alain DAGHER, Montreal (CA); Marcel FARRES-FRANCH, Montreal (CA)

(73) Assignees: Ichilov Tech Ltd., Tel-Aviv (IL); Ramot at Tel-Aviv University Ltd., Tel-Aviv (IL); The Royal Institution For The Advancement Of Learning/McGill University, Montreal (CA)

(21) Appl. No.: 18/085,700

(22) Filed: Dec. 21, 2022

Related U.S. Application Data
(63) Continuation of application No. PCT/IL2021/050764, filed on Jun. 22, 2021.
(60) Provisional application No. 63/042,404, filed on Jun. 22, 2020.

(51) Int. Cl. A61B 5/375; A61B 5/38; A61B 5/372; G16H 20/30; G01R 33/48

(52) U.S. Cl. CPC A61B 5/375; A61B 5/38; A61B 5/372; G16H 20/30; G01R 33/4806

(57) ABSTRACT

A neurofeedback method, including:
recording electrical signals from at least one brain region of a subject, wherein changes in the recorded electrical signals over time indicate changes in an activity level of the at least one brain region;
providing an audio signal having a perceived quality based on the recorded electrical signals and
according to an activity level of the at least one brain region;
delivering the audio signal to the subject during said recording.

### RELATED APPLICATIONS

This application is a Continuation (CON) of PCT Patent Application No. PCT/IL2021/050764 having International filing date of Jun. 22, 2021, which claims the benefit of priority of U.S. Provisional Patent Application No. 63/042,404 filed on Jun. 22, 2020. The contents of the above applications are all incorporated by reference as if fully set forth herein in their entirety.

### FIELD AND BACKGROUND OF THE INVENTION

The present invention, in some embodiments thereof, relates to modulating an activity of a mesolimbic brain region and, more particularly, but not exclusively, to modulating an activity of the ventral striatum brain region.

Recent advances in computational abilities have opened a path to selectively monitor a particular brain region with relatively fine spatial resolution via real-time functional Magnetic Resonance Imaging (rt-fMRI). This has enabled the development of several Brain-computer-interface (BCI) approaches, such as neurofeedback—a particular form of bio-feedback in which the feedback provided to participants is derived from brain signals obtained continuously. Such learnt rt-fMRI-NF modulation of deep brain regions, such as the amygdala, was found to be effective in reducing depressive symptoms in MDD.

While opening an exciting new avenue for non-invasive “cognitive neurostimulation”, the utility of rt-fMRI-NF for neuromonitoring is considerably limited due to immobility, high-cost and extensive physical requirements of the scanning procedure. Electroencephalography (EEG), on the other hand, is low-cost and accessible, and thus adjusted for repeated and/or home-based monitoring. However, EEG suffers from poor spatial resolution that especially hampers the targeting of deep brain areas such as in the mesolimbic pathway.

When aiming to target a reward-specific system, it seems important to rely on a reliable neural indicator for reward-related processes in deep brain areas such as the Ventral Striatum (VS) and/or Ventral tegmental area (VTA) to achieve the necessary functional outcomes. To overcome this difficulty, it is possible to enhance the spatial localization of EEG using computational tools. A few attempts were made in this direction using Low-Resolution Electromagnetic Tomography (LORETA)(Grech et al., 2008) or its variants (Congedo, Lubar, & Joffe, 2004; Thatcher, 2010). However, this approach necessitates the use of a dense grid of electrodes, limiting the potential mobility and accessibility of this method. In addition, it is sensitive to noise, especially in deep subcortical areas and still has relatively low spatial resolution (Yao & Dewald, 2005). Theory driven approaches that utilized fMRI to improve EEG localization attempted to construct a forward model that traces neuronal activity from both measures (Valdes-Sosa et al., 2009). However, such a model-based approach relies on a-priori assumptions regarding the biophysical origins of the EEG and fMRI signals.

To overcome the lack of a-priori knowledge about the biophysical origins, it is possible to apply data-driven approaches to associate between the two signal-types (Laufs, Daunizeau, Carmichael, & Kleinschmidt, 2008; Meir-Hasson, Kinreich, Podlipsky, Hendler, & Intrator, 2014; Valdes-Sosa et al., 2009). Earlier studies using such an approach have used correlations to explore the link between particular EEG frequency bands, such as alpha (9-13 Hz), and localized BOLD activity (e.g., Ben-Simon, Podlipsky, Arieli, Zhdanov, & Hendler, 2008; de Munck et al., 2007; Goldman, Stern, Engel Jr, & Cohen, 2002).

Later research demonstrated that linear regression using a combination of frequency-bands predicts localized BOLD activity better than individual bands (Mantini, Perrucci, Del Gratta, Romani, & Corbetta, 2007; Zumer, Brookes, Stevenson, Francis, & Morris, 2010). Nevertheless, these described methods necessitate the simultaneous use of fMRI and EEG, thus limiting their accessible utilization for repeated NF training.

Most recently, a statistical-modeling based framework, which utilizes machine-learning methods for generating an fMRI-inspired EEG model of the BOLD activation within a particular region or network was developed. The modeling of the EEG relies on multivariate time and frequency information and can be applied at the level of a single electrode. Using such a model, termed electrical finger print: EFP, Meir-Hasson et al. were able to predict fMRI activation of a deep brain region using EEG data. The model presented was based on weights of different frequency bands and their associated time delays, enabling to predict BOLD signal in the targeted region using EEG alone. The fingerprinting approach was realized recently by constructing an fMRI-based EEG model of a deep brain structure—the amygdala (Meir-Hasson et al., 2016; Meir-Hasson et al., 2014)—and then used within a neurofeedback (NF) procedure, yielding a real-time EEG technique that is based on an fMRI probe of amygdala activation (Cavazza et al., 2014; Cohen et al., 2016; Keynan et al., 2016; Meir-Hasson et al., 2016).

Results from validation experiments of this method indicated that subjects who were trained outside the fMRI-scanner to down-regulate the amygdala-EFP not only successfully decreased amygdala BOLD activity during fMRI-NF in a later session (Keynan, 2016; 2019), but also manifested reduced amygdala reactivity to threatening visual stimuli, as compared to subjects who underwent sham-EFP-NF. Moreover, amygdala-EFP-NF resulted in improved performance in a task that examines implicit emotion regulation (Keynan et al., 2016) and has been shown to be applicable in clinical contexts (i.e., Fibromyalgia; Goldway, NIMG, 2019). Finally, analysis of the EFP-BOLD correlates has revealed that the amygdala-EFP signal correlated with BOLD activity in the right amygdala (Keynan et al., 2016).

### SUMMARY OF THE INVENTION

Some examples of some embodiments of the invention are listed below:

Example 1. A neurofeedback method, comprising:

recording electrical signals from at least one brain region of a subject, wherein changes in said recorded electrical signals over time indicate changes in an activity level of said at least one brain region;

providing an audio signal having a perceived quality based on said recorded electrical signals and according to an activity level of said at least one brain region;

delivering said audio signal to the subject during said recording.

Example 2. A method according to example 1, comprising degrading said audio signal prior to said delivering.

Example 3. A method according to example 2, wherein said degrading comprises reducing a perceived quality of said audio signal.

Example 4. A method according to any one of examples 2 or 3, comprising instructing said subject to change said degrading.

Example 5. A method according to any one of examples 3 or 4, comprising changing said degradation according to said changes in an activity level of said at least one brain region.

Example 6. A method according to any one of examples 2 to 5, wherein said audio signal comprises music, and wherein said degrading comprises degrading a perceived quality of said music.

Example 7. A method according to example 6, wherein said music is a music selected by the subject as a pleasurable music.

Example 8. A method according to any one of examples 6 or 7, wherein said music is a music affecting mood in said subject.

Example 9. A method according to any one of examples 6 to 8, wherein said at least one brain region is a brain region having an activity that is affected by application of said music.

Example 10. A method for determining an activity level of the ventral striatum (VS), comprising:

providing a fingerprint indicating a relation between measured electrical signals and an activity level of said VS;

positioning at least one electrode on a scalp of a subject according to said fingerprint;

recording and processing electrical signals received from said at least one electrode according to said fingerprint;

determining an activity level of said VS according to said processed electrical signals.

Example 11. A method according to example 10, comprising:

determining a correlation between said processed electrical signals and said fingerprint, and wherein said determining comprises determining an activity level of said VS according to said determined correlation.

Example 12. A method according to any one of examples 10 and 11, wherein said electrical signals comprise EEG signals, and wherein said fingerprint indicates a relation between processed EEG signals and an activity level of said VS.

Example 13. A method according to any one of examples 10 to 12, wherein said positioning comprises positioning the at least one electrode in one or more locations including C4, F7, F8, T7, T8, P8, TP9 and TP10 of an EEG positioning system.

Example 14. A method according to any one of examples 10 to 13, wherein said provided fingerprint is a multi-dimensional model generated by correlating EEG data and fMRI-BOLD activity of the VS, wherein said multi-dimensional model comprises a coefficient matrix corresponding to frequency bands, electrodes and one or more time windows.

Example 15. A method according to example 14, wherein said one or more time windows comprises a time window of up to 30 seconds.

Example 16. A method for treating Anhedonia, comprising:

diagnosing a subject with Anhedonia;

identifying one or more tasks shown to increase activity level of the ventral striatum in said subject;

instructing said subject to perform said one or more tasks.

Example 17. A method according to example 16, wherein said diagnosing comprises determining an activation level of at least one specific brain region of a reward system, and diagnosing said subject with anhedonia if said determined activation level is lower than a predetermined activation level.

Example 18. A method according to example 17, wherein said diagnosing comprises delivering a stimulus to said subject selected to increase an activation level of the at least one specific brain region, and wherein said diagnosing comprises diagnosing said subject with anhedonia if a response of said subject to said delivered stimulus is lower than a predetermined response, based on said determined activation.

Example 19. A method for treating Apathy, comprising:

diagnosing a subject with Apathy;

identifying one or more tasks shown to increase activity level of the ventral striatum in said subject;

instructing said subject to perform said one or more tasks.

Example 20. A method according to example 19, wherein said diagnosing comprises determining an activation level of at least one specific brain region of a reward system, and diagnosing said subject with apathy if said determined activation level is lower than a predetermined activation level.

Example 21. A method according to example 20, wherein said diagnosing comprises delivering a stimulus to said subject selected to increase an activation level of the at least one specific brain region, and wherein said diagnosing comprises diagnosing said subject with apathy if a response of said subject to said delivered stimulus is lower than a predetermined response, based on said determined activation.

Example 22. A method for treating a subject with Anhedonia, comprising:

recording electrical signals from a brain of a subject diagnosed with Anhedonia;

determining an activity level of the ventral striatum (VS) using said recorded electrical signals;

generating a human detectable indication according to said determined activity level;

delivering said human detectable indication to said subject during said recording;

instructing said subject to perform at least one mental exercise shown to increase the activity level of the VS;

modifying said human detectable indication to a more pleasurable indication if activity level of said VS is increased.

Example 23. A method according to example 22, comprising:

determining a desired level of said VS.

Example 24. A method according to any one of examples 22 or 23, wherein said modifying comprises modifying said human detectable indication during said delivering.

Example 25. A method according to any one of examples 22 to 24, wherein said human detectable indication comprises an audio indication or a visual indication.

Example 26. A neurofeedback method, comprising:

recording electrical signals from at least one specific deeply located brain region of a subject, wherein changes in said recorded electrical signals over time indicate changes in an activity level of said at least one brain region;
identifying an increase in activation of said at least one specific brain region based on the recorded electrical signals;
delivering a positive feedback signal to said subject according to said identified increase in activation of said at least one brain region, during said recording.

Example 27. A method according to example 26, wherein said delivering of said positive signal comprises improving a quality of a feedback signal delivered to said subject according to said identified increase in activation of said at least one brain region, during said recording.

Example 28. A method according to example 27, wherein said feedback signal comprises a music feedback signal, and wherein said improving comprises improving a quality of said music feedback signal according to said identified increase in activation of said at least one brain region during said recording.

Example 29. A method according to any one of examples 26 to 28, wherein said recording comprises recording EEG electrical signals, and wherein said identifying comprises determining a relation between at least a portion of said recorded EEG electrical signals and at least one electrical fingerprint indicating a specific activation level of said at least one specific brain region.

Example 30. A method according to example 29, wherein said at least one electrical fingerprint indicates a specific previously measured fMRI-BOLD activity of said at least one specific brain region.

Example 31. A method according to any one of examples 26 to 30, wherein said at least one specific deeply located brain region comprises a mesolimbic brain region and/or a brain region of a reward system.

Example 32. A method according to example 31, wherein said mesolimbic brain region and/or said brain region of the reward system, comprise a ventral striatum (VS), a ventromedial prefrontal cortex (vMPFC), and an anterior mid cingulate cortex (aMcc), and/or anterior insula.

Example 33. A neurofeedback system, comprising:

at least one electrode for recording electrical signals from a subject brain;
memory which stores at least one electrical fingerprint indicating an activity level of at least one deeply located brain region of a mesolimbic system and/or of a reward system;
a user interface configured to generate and deliver a feedback signal to said subject;
a control circuitry configured to;

receive electrical signals recorded by said at least one electrode;

identify a correlation between at least a portion of said recorded electrical signals and said at least one electrical fingerprint;

determine an activation level of said at least one deeply located brain region based on said identified correlation; and

signal said user interface to deliver a positive feedback signal to said subject when an increase in activity of said at least one deeply located brain region is determined.

Example 34. A system according to example 33, wherein said positive feedback signal is a feedback signal configured to trigger said subject to increase an activity level of said at least one deeply located brain region.

Example 35. A system according to any one of examples 33 or 34, wherein said stored at least one electrical fingerprint comprises a multi-dimensional model generated by correlating EEG data and fMRI-BOLD activity of the VS, wherein said multi-dimensional model comprises a coefficient matrix corresponding to frequency bands, electrodes and one or more time windows.

Example 36. A system according to example 35, wherein said one or more time windows comprises a time window of up to 30 seconds.

Example 37. A system according to any one of examples 33 to 36, wherein said control circuitry is configured to signal said user interface to degrade a feedback signal and deliver the degraded feedback signal to said subject prior to receiving said electrical signals.

Example 38. A system according to example 37, wherein said control circuitry signals said user interface to generate said positive signal by increasing a quality of said degraded feedback signal.

Example 39. A method for treating a subject having a dysfunctional reward system, comprising:

providing a stimulus to said subject, wherein said stimulus is selected to affect an activity of at least one specific brain region of a reward system;

determining an activity level of said at least one specific brain region;

modifying said stimulus if said activity of said at least one specific brain region is increased according to results of said determining.

Example 40. A method according to example 39, wherein said stimulus comprises a degraded stimulus, and wherein said modifying comprises modifying a degradation of said degraded stimulus if said activity of said at least one specific brain region is increased according to results of said determining.

Example 41. A method according to example 40, wherein said modifying comprises improving a quality of said degraded stimulus if said activity of said at least one specific brain region is increased according to results of said determining.

Example 42. A method according to example 40, wherein said modifying comprises reducing a quality of said degraded stimulus if said activity of said at least one specific brain region is increased according to results of said determining.

Example 43. A non-volatile memory having stored therein a model linking EEG measurements to a fMRI-BOLD signal indicating a selective activation of the Ventral Striatum (VS).

Example 44. A non-volatile memory according to example 43, wherein said stored model comprises a coefficient matrix of at least 100 coefficients corresponding to frequency bands, electrodes and one or more time windows.

Example 45. A non-volatile memory according to example 44, wherein said electrodes comprise one or more electrodes in locations C4, F7, F8, T7, T8, P8, TP9 and TP10 of an EEG positioning system.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such as generating an electrical fingerprint might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a schematic representation of a process for generating a signature of the Ventral Striatum (VS) activity, according to some exemplary embodiments of the invention;

FIG. 1B is a heat map showing an example of a VS signature, according to some exemplary embodiments of the invention;

FIG. 1C is a flow chart of a process for determining an activity of a brain region of the mesolimbic system, according to some exemplary embodiments of the invention;

FIG. 1D is a flow chart of a process for delivering a positive feedback signal when identifying an increase in activation of a deeply located brain region, according to some exemplary embodiments of the invention;

FIG. 1E is a flow chart of a process for increasing a quality of a degraded feedback signal when identifying an increase in activation of a deeply located brain region, according to some exemplary embodiments of the invention;

FIG. 1F shows reward domain engagement, as demonstrated in a validation and feasibility experiment;

FIG. 1G shows an evaluation of a fingerprint model, as demonstrated using two validation approaches (leave-one out validation applied on the modeling dataset and external validation applied on an independent replication dataset, as demonstrated in a validation and feasibility experiment;

FIG. 1H shows an evaluation of the fingerprint model performance in a different reward context, as demonstrated in a validation and feasibility experiment;

FIG. 1I shows music reward related modulation of the VS-EFP fingerprint, as demonstrated in a validation and feasibility experiment;

FIG. 1J shows the use of the VS-EFP fingerprint in neurofeedback context, as demonstrated in a validation and feasibility experiment;

FIG. 2 is a schematic representation of a neurofeedback process using a music interface, according to some exemplary embodiments of the invention;

FIGS. 3A and 3B are schematic representations of a study design for validating upregulation of the ventral striatum;

FIGS. 4A and 4B are graphs showing modulation of a ventral striatum fingerprint during the validation study;

FIG. 5A is an fMRI image showing activation of the ventral striatum during the validation study;

FIG. 5B is a graph showing regulation of the left and right ventral striatum during the validation study and according to some exemplary embodiments of the invention;

FIG. 5C is a graph showing change in VS-BOLD self-regulation per group, as shown in the validation study;

FIG. 6A is a graph showing an effect of ventral striatum training on reward-based learning during the validation study and according to some exemplary embodiments of the invention;

FIG. 6B is a schematic illustration showing results of a probabilistic selection task during the validation study;

FIG. 6C is a block diagram of a system for delivery of a neurofeedback-related process, according to some exemplary embodiments of the invention;

FIG. 7A is a graph showing modulation of VS-EFP per group and session of a neurofeedback process relative to the first session, as demonstrated in a neurofeedback proof of concept validation experiment;

FIG. 7B is a graph showing neurofeedback performance in improvement of maximal VS-EFP modulation relative to the first session in the control and test groups per session of the neurofeedback proof of concept validation experiment;

FIGS. 8A-8B are graphs showing association between neurofeedback training and changes in reward related behavior, as demonstrated in a neurofeedback proof of concept validation experiment;

FIG. 9 includes graphs showing a correlation between success in neurofeedback performance using VS-EFP in the last session and measure of anhedonia following training compared to a control group, as demonstrated in a neurofeedback proof of concept validation experiment; and

FIG. 10 is a graph showing changes in positive affect at the beginning of each neurofeedback training session during neurofeedback using VS-EFP compared to a control group, as demonstrated in a proof of concept neurofeedback validation experiment.

### DESCRIPTION OF SPECIFIC EMBODIMENTS OF THE INVENTION

The present invention, in some embodiments thereof, relates to modulating an activity of a mesolimbic brain region and, more particularly, but not exclusively, to modulating an activity of the ventral striatum brain region.

An aspect of some embodiments relates to providing a neurofeedback to a subject by providing an audio signal having a perceived quality according to an activity level of at least one brain region of a subject. In some embodiments, the brain activity is determined by recording electrical signals from the subject, and the audio signal is generated based on the recorded electrical signals. In some embodiments, the audio signal comprises music. In some embodiments, the audio signal is delivered to the subject.

According to some embodiments, the audio signal is degraded, for example before it is provided to the subject. In some embodiments, degradation of the audio signal comprises reducing a perceived quality of the audio signal. In some embodiments, the subject is instructed to change the degradation of the audio signal, for example the subject is instructed to perform a task, for example a mental or a cognitive task shown to affect the degradation of the audio signal.

According to some embodiments, the degradation is changed according to changes in the activity level of the at least one brain region, for example the VS. In some embodiments, when the audio signal comprises music, degrading comprises degrading or reducing a perceived quality of the music. In some embodiments, the music is a music selected by the subject to be a pleasurable music. In some embodiments, the music is a music affecting the mood of the subject. In some embodiments, the at least one brain region is brain region affected by application of the audio signal, for example application of the music.

An aspect of some embodiments relates to delivering a neurofeedback procedure, for example neurofeedback training or neurofeedback treatment, to a subject, by modifying a quality of a feedback signal provided to the subject. In some embodiments, the quality of the provided feedback signal is improved, according to a change in activation of at least one specific brain region, for example a change in activation at a desired direction. As used herein, a specific brain region means a brain region having a volume which is less than 25%, for example less than 20%, less than 15%, less than 10%, less than 5% or any intermediate, smaller or larger percentage value, from a total volume of a brain of a subject, for example a human subject.

According to some embodiments, at least one specific brain region is a deeply located brain region. In some embodiments, the at least one specific brain region comprises a brain region of the mesolimbic system or at least one specific brain region of the reward system. In some embodiments, the at least one specific brain region of the mesolimbic system comprises the VS.

According to some embodiments, the quality of the provided feedback signal is improved when an activation of the at least one specific brain region is increased. Alternatively, the quality of the provided feedback signal is improved when an activation of the at least one specific brain region is reduced. Alternatively, the quality of the feedback signal is degraded if an activation of the at least one specific brain region is reduced.

In some embodiments, the feedback signal is delivered online while monitoring the activity level of the at least one specific brain region. In some embodiments, the feedback signal is modified online, for example while monitoring the activity level of the at least one specific brain region. In some embodiments, the feedback signal is provided continuously. Alternatively, the feedback is provided at the end of each regulation block, as an intermittent feedback. Optionally, only positive or only negative feedback may be provided. In some embodiments, the activity of the at least one specific brain region is monitored based on EEG signals recorded from the at least one specific brain region without a need for spatial scan data, for example fMRI data.

According to some embodiments, the feedback signal comprises music. In some embodiments, at least one parameter of the music is modified according to an activity level of the at least one specific brain region. In some embodiments, a volume of the music signal is increased according to an increase in the activation of the at least one specific brain region. Alternatively or additionally, a distortion level, for example a degradation level of the music signal provided as feedback is reduced when an activity level of the at least one specific brain region is elevated.

An aspect of some embodiments relates to increasing an activity of at least one specific brain region in a subject brain, for example a deeply located brain region by delivering a positive feedback to the subject. In some embodiments, the positive feedback is delivered to the subject online while monitoring the activity of the at least one specific brain region, for example based on recorded EEG signals and optionally without a need to use spatial scan data, for example fMRI data. In some embodiments, the positive feedback is delivered to the subject when activity of the at least one specific brain region is increased.

According to some embodiments, the positive feedback is provided by modifying a feedback interface in a way that encourages said subject to continue to increase the activity of the at least one specific brain region. In some embodiments, the positive feedback is provided continuously, for example when the activity of the at least one specific brain region is increased. In some embodiments, the positive feedback comprises improving a quality of the feedback interface according to an increase in activity of the at least one specific brain region.

According to some embodiments, improving a quality of the feedback interface comprises improving a quality of an audio and/or a visual signal provided to a subject.

An aspect of some embodiments relates to an electrical fingerprint (EFP) based on EEG signals that correlates with fMRI-B OLD activity of one or more specific brain regions of the mesolimbic system, for example the VS. In some embodiments, the one or more specific brain regions of the mesolimbic system comprise deeply located brain regions, for example ventromedial prefrontal cortex (vMPFC), anterior midcingulate cortex (aMcc), and/or anterior insula. In some embodiments, the electrical fingerprint is a process-specific fingerprint, generated while one or more subjects are engaged in tasks that are known to affect the reward system.

According to some embodiments, the fingerprint is a model linking EEG measurements to a fMRI-B OLD signal indicating a selective activation of at least one specific brain region, for example the Ventral Striatum (VS). As used herein, a selective activation of a brain region means activation of the at least one specific brain region in a level that is higher from activation levels of other brain regions, for example more than 30% of other brain regions, for example more than 50% of other brain regions, more than 60% of other brain regions, more than 80% of other brain regions, more than 90% of other brain regions.

According to some embodiments, the model comprises a coefficient matrix of at least 100 coefficients corresponding to frequency bands, electrodes and one or more time windows.

According to some embodiments, the EFP comprises electrical signals, for example EEG electrical signals recorded from EEG electrodes located at positions C4, F7, F8, T7, T8, P8, TP9 and TP10. In some embodiments, the EFP comprises EEG electrical signals in a frequency range between 0-40 Hz, and in a time delay window between 0 and 30 seconds.

An aspect of some embodiments relates to monitoring the activity level of the ventral striatum (VS) using EEG signals without the need of imaging analysis. In some embodiments, the activity level of the VS is monitored using at least one fingerprint which indicates a relation between measured electrical signals and an activity level of the VS. In some embodiments, the fingerprint is an electrical fingerprint (EFP), for example as described in WO2012/104853 and in U.S. patent application Ser. No. 13/983,419.

According to some embodiments, electrodes, for example EEG electrodes are positioned on a scalp of a subject according to the EFP. In some embodiments, electrical signals are recorded and processed according to the fingerprint. In some embodiments, an activity level of the VS is determined according to the processed signals. In some embodiments, recording and processing of an electrical signals, and determining an activity level of the VS are performed as described in WO2012/104853 and in U.S. patent application Ser. No. 13/983,419.

According to some embodiments, a correlation is determined between the processed electrical signals and the fingerprint. In some embodiments, an activity level of the VS is determined according to the correlation.

An aspect of some embodiments relates to treating Anhedonia in a patient, or in a healthy subject having a train of anhedonia, by increasing the activity of the VS in the patient or by increasing a potential of a patient to self regulate, for example up-regulate, the patient VS. In some embodiments, the activity of the VS is increased by instructing the patient to perform at least one task, for example a mental or a motoric task. Alternatively, the activity of the VS is increased without providing instructions to the patient, for example via implicit regulation. In some embodiments, the task was previously shown to increase the activity of the VS in this specific patient.

As used herein a task which increases the activity of the VS is any task that increases the activity is any task that increases the activity of the reward system, for example a game, a rewarding game, a task that promotes recollection of a good memory, for example by presenting an image of a beloved person and/or presenting a picture of a public figure, a task that includes solving a problem and/or listening to pleasurable music.

According to some embodiments, electrical signals are recorded from a patient diagnosed with Anhedonia. In some embodiments, an activity level of the VS is determined using the recorded electrical signals. In some embodiments, a human detectable indication is generated according to the determined activity level. In some embodiments, the human detectable indication comprises at least one of an audio signal, a visual signal, music, and a picture. In some embodiments, the human detectable indication is delivered to the subject, for example during the recording of the electrical signals, for example as an online continuous feedback. In some embodiments, the subject is instructed to perform at least one task, for example a mental task that was previously shown to increase the activity levels of the VS. Optionally, the indication is modified to a more pleasurable indication if an activity level of the VS is increased.

According to some embodiments, a desired level of the VS, for example a desired activity pattern of the VS is predetermined. In some embodiments, the indication is modified during the delivery.

An aspect of some embodiments relates to treating Apathy in a patient or a healthy subject having a trait of apathy, by increasing the activity of the VS in the patient. In some embodiments, the activity of the VS is increased by instructing the patient to perform at least one task, for example a mental or a motoric task. In some embodiments, the task was previously shown to increase the activity of the VS in this specific patient.

According to some embodiments, a specific electrical fingerprint (EFP) is generated, for the VS. In some embodiments, the EFP fingerprint links electrical signals, for example EEG electrical signals or processed EEG electrical signals with a specific activity level of the VS. In some embodiments, using the EFP, electrical signals, for example EEG signals recorded from a subject are processed, and based on the EFP an activity level of the VS is determined.

According to some embodiments, the activity level of the VS is monitored and/or modified, for example when a treatment of a mental condition or a mental disease is directed to increase the activity of the reward system. In some embodiments, the activity level of the VS is monitored when treating a patient diagnosed with Apathy or Anhedonia.

According to some embodiments, a neurofeedback (NF) treatment is delivered to a subject as part of a treatment for modulating, for example increasing, the activity of the VS and/or the reward system. In some embodiments, modulating an activity of a brain region means modulating an electrical fingerprint of the brain region. Alternatively or additionally, the NF treatment is delivered to a subject as part of a treatment for improving an ability to self-regulate, for example upregulate of the VS and/or the reward system. In some embodiments, a feedback regarding the activity of the VS is delivered while the subject performs a task that is predicted to increase the activity of the VS or the activity of the reward system. In some embodiments, the feedback is based on delivery of an audio signal to the subject, for example in the form of music. Alternatively or additionally, the feedback is provided as a visual signal, for example a picture. In some embodiments, the signal, for example the audio signal or the visual signal is degraded. In some embodiments, the subject is requested to try and modify the degradation of the signal into a more pleasurable signal. In some embodiments, the degradation process of the signal depends on a current activity level of the brain region, for example the VS, and a desired activity level of the VS.

According to some embodiments, when the subject succeeds in increasing or decreasing the activity level to a desired activity level, the signal becomes less degraded, for example more pleasurable. In some embodiments, the degradation level of the signal, for example the audio signal or the visual signal, is correlated with the activity of the brain region, and is optionally serves as a continuous or on-line feedback to the subject while the subject tries to modulate the activity of the brain region.

According to some embodiments, this type of a neurofeedback system, where changes in degradation of a signal delivered to a subject provide a feedback regarding an activity level of the VS, is used when treating Anhedonia, Apathy.

According to some embodiments, the neurofeedback process described in this application can be used for treating healthy human subjects having high levels of anhedonia and/or apathy trait. In some embodiments, the neurofeedback process is similar to the treatment method used to treat subjects diagnosed with apathy or anhedonia.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### Exemplary Ventral Striatum Fingerprint

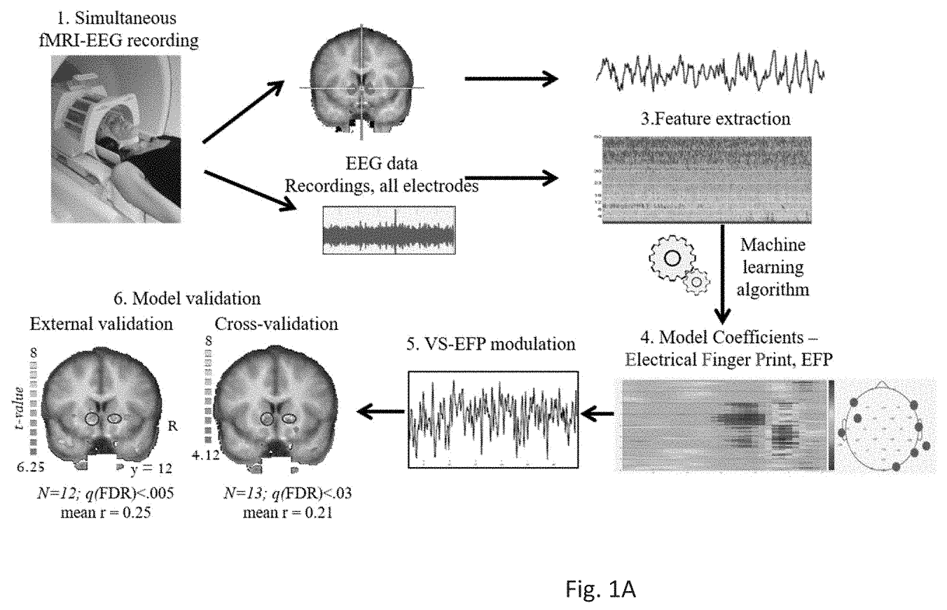

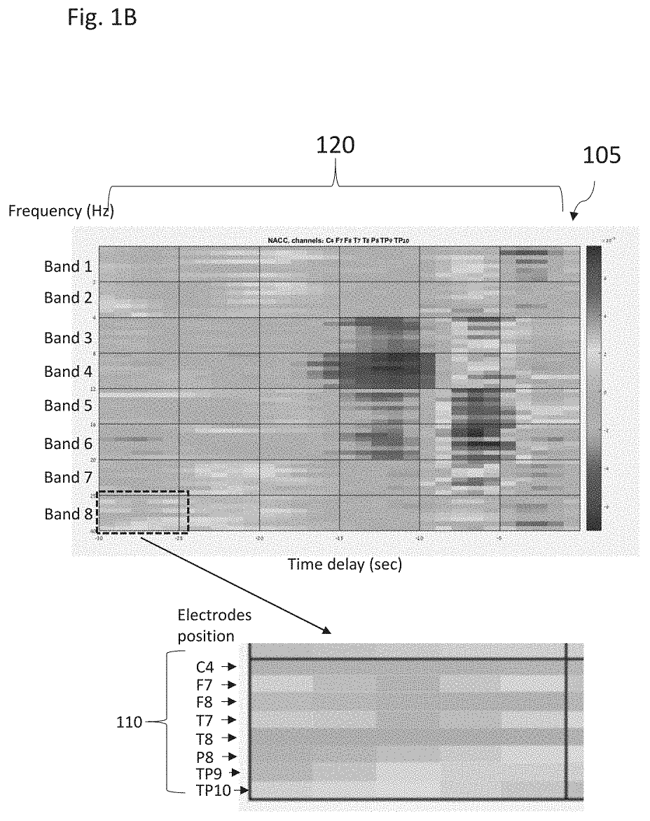

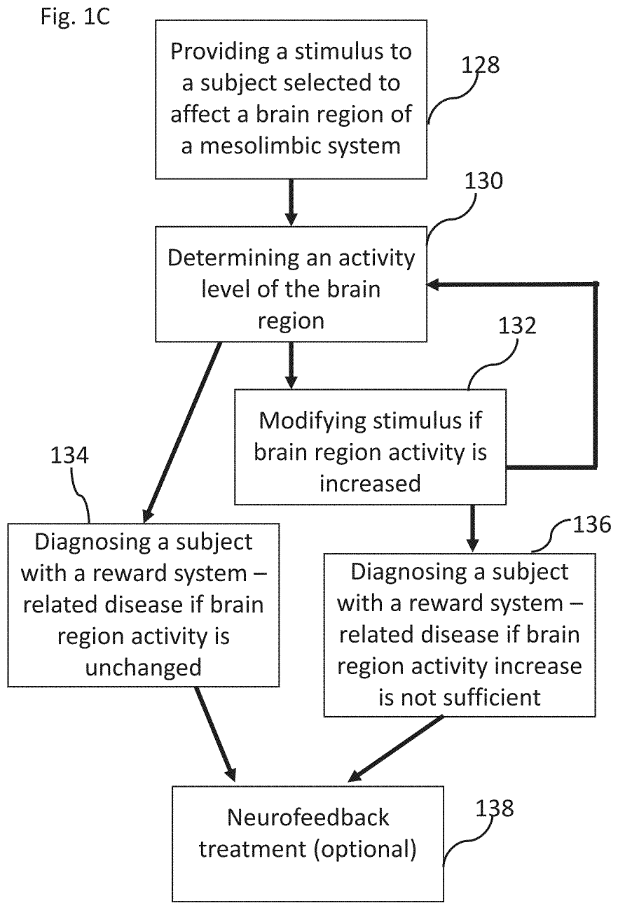

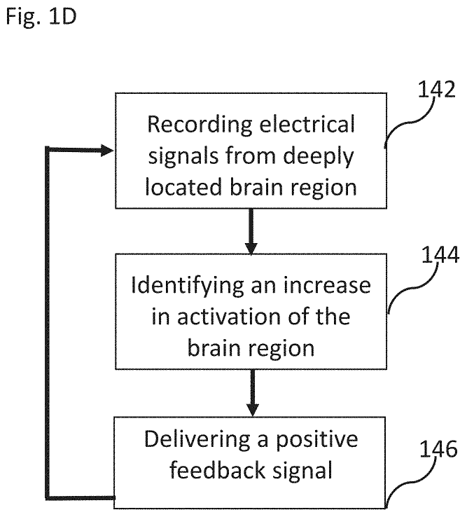

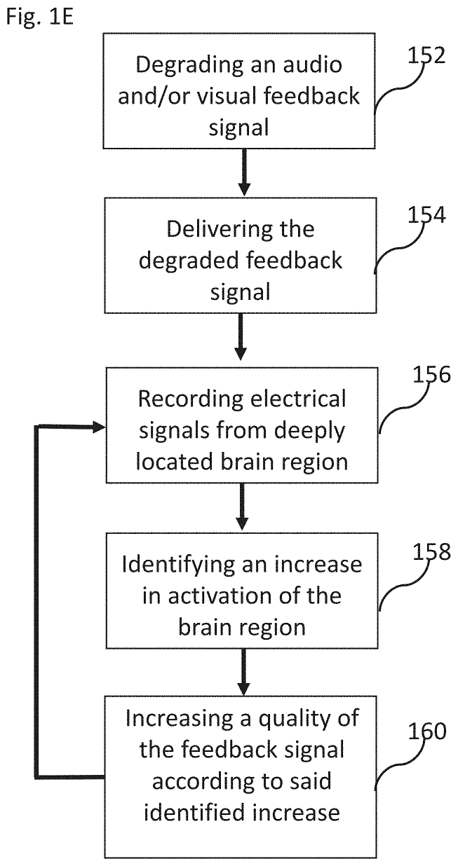

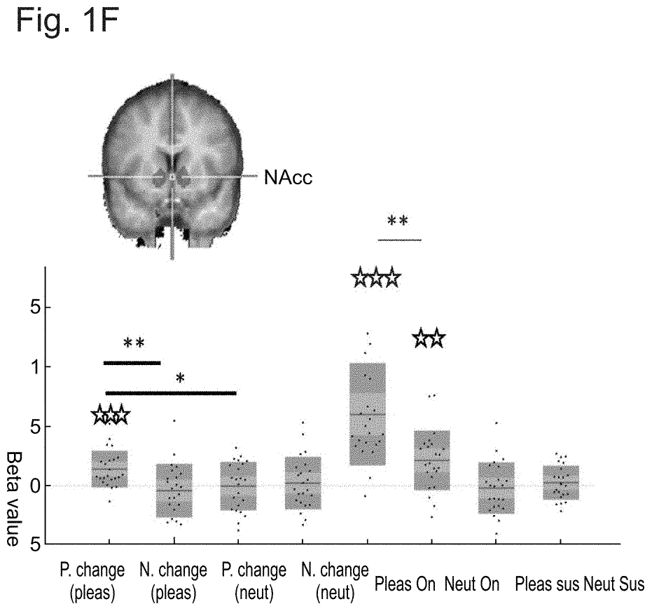

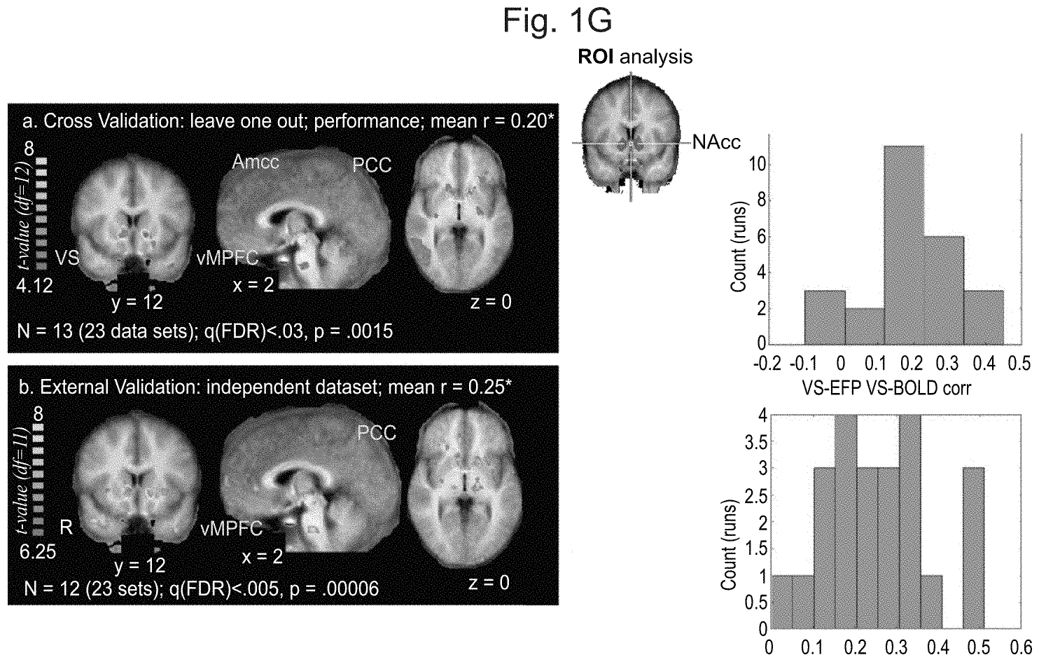

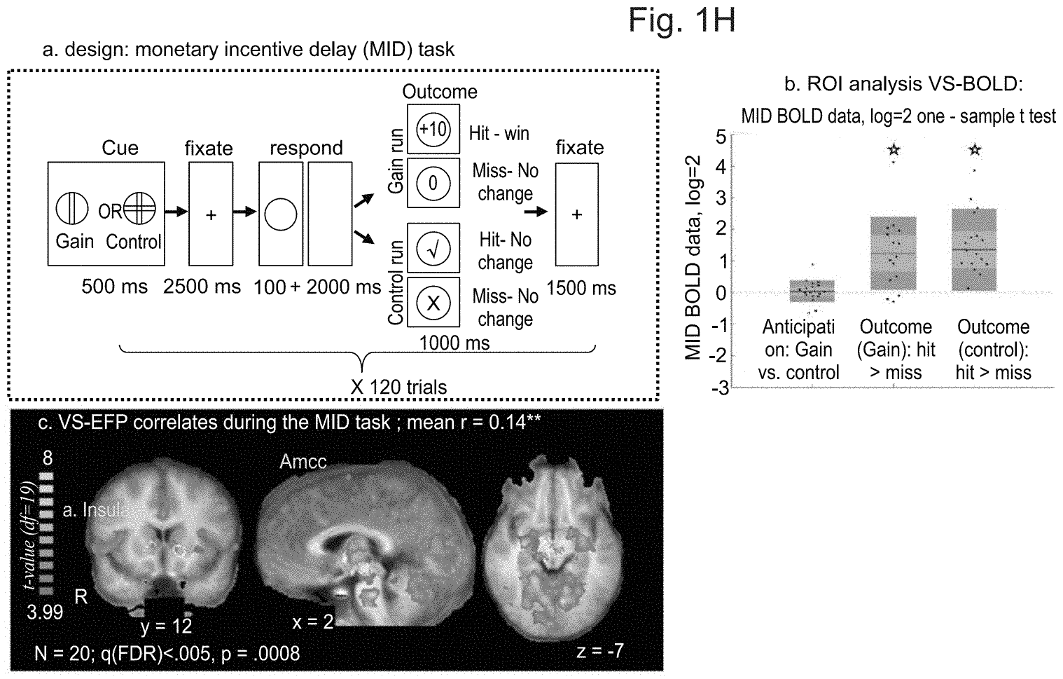

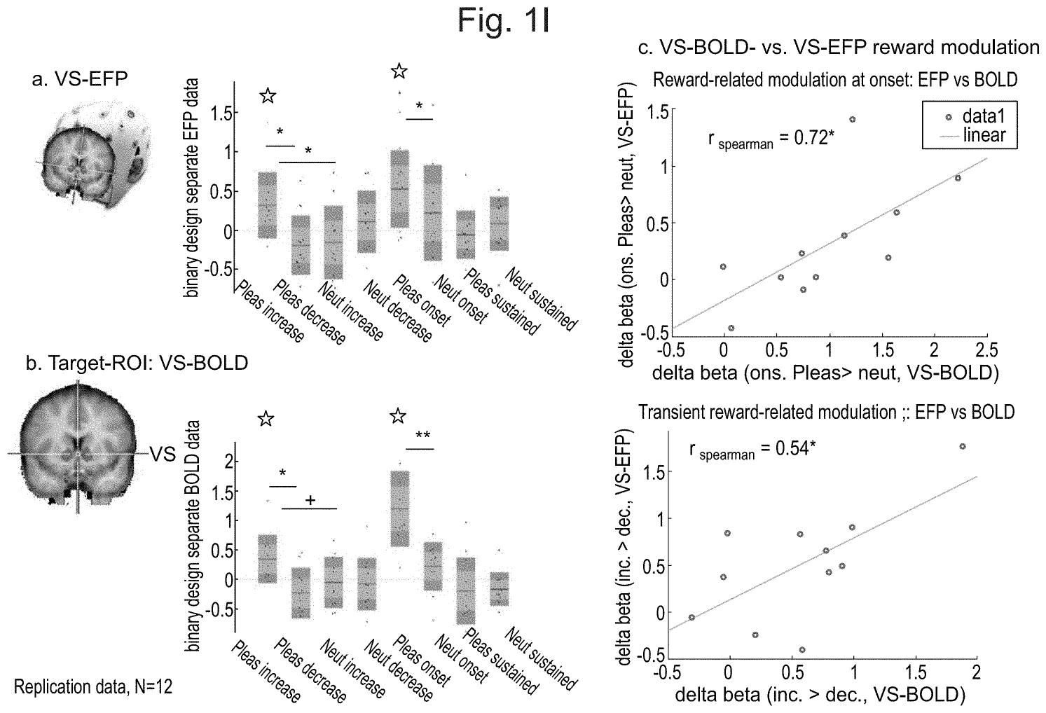

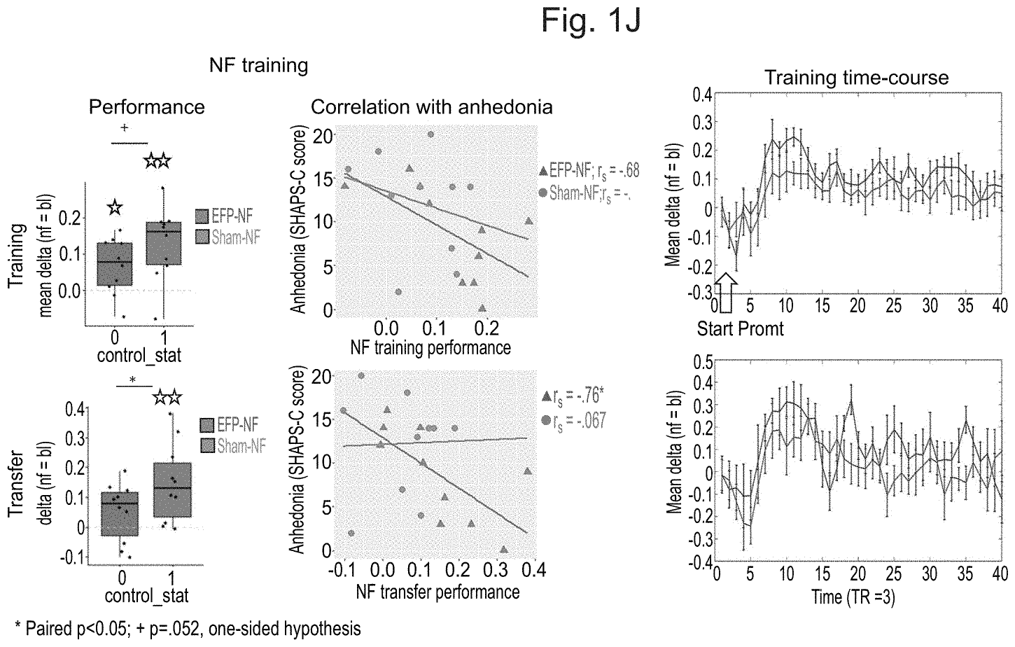

Reference is now made to FIG. 1A, depicting a process for generating a signature, for example a fingerprint, of the Ventral Striatum (VS) activity, according to some exemplary embodiments of the invention.

According to some exemplary embodiments, a machine learning-based approach to predict BOLD activity in a predefined region of interest using simultaneously acquired EEG is applied in order to generate a VS electrical fingerprint (VS-EFP). In some embodiments, for example as shown in step (1), EEG and fMRI data are acquired simultaneously, for example during a music listening task from 30 participants in two scanning batches of 15 subjects each. In some embodiments, during step (2) the fMRI time course and the (3) time-frequency matrix obtained from the EEG data are used to calculate the model. In some embodiments, in step (4) the model's coefficient matrix is applied on the EEG data to construct the (5) VS-EFP time-courses. In some embodiments, the resulting time courses are used as regressors to assess the model's performance via whole-brain random effects general liner model analysis in two different datasets.

Reference is now made to FIG. 1B, depicting an example of a VS fingerprint, for example an according to some exemplary embodiments of the invention.

According to some exemplary embodiments, a fingerprint 105 is in a form of a heat map, where the y-axis indicates a range of frequencies, for example from 0 to 40 Hz, and the x-axis indicates time delay, for example between 0 and 30 seconds. In some embodiments, the range of frequencies is divided into one or more band of frequencies, where each frequency band represents a sub-range of frequencies. For example, as shown in FIG. 1B, the frequency range of 0-40 Hz is divided into 8 bands, for example 8 power bands, where band 1 indicates a frequency range between 0-2 Hz, band 2 indicates a frequency range between 2-4 Hz, band 3 indicates a frequency range between 4-8 Hz, band 4 indicates a frequency range between 8-12 Hz, band 5 indicates a frequency range between 12-16 Hz, band 6 indicates a frequency range between 16-20 Hz, band 7 indicates a frequency range between 20-25 Hz, and band 8 indicates a frequency range between 25-40 Hz. In some embodiments, the total frequency range is divided to a smaller or larger number of frequency bands. In some embodiments, each frequency band indicates a different, for example, a smaller or larger frequency range.

According to some exemplary embodiments, each of the horizontal lanes 110, for example in each frequency band, represents recordings of an EEG electrode located at a different position on a scalp of a subject. For example, EEG electrodes located at positions C4, F7, F8, T7, T8, P8, TP9, and TP10. In some embodiments, each of the vertical columns 120 divides the time delay range of the x-axis into sub-ranges of time delays in the recordings of each electrode and at specific range of frequencies.

In some embodiments, a different number of electrodes is used and/or electrodes at different positions to generate the EFP signature. In some embodiments, the colors of the heat map represent a power or an intensity of the signal of a recorded signal.

According to some exemplary embodiments, a fingerprint for the VS may vary in up to 10%, up to 15%, up to 20% from the fingerprint 105 shown in the heat map. In some embodiments, a fingerprint for VS may include fingerprints in which the delays 120 move in time up to 5%, 10%, 15 forward or backward in time, relative to fingerprint 105. In some embodiments, a fingerprint comprises 20%, 30%, 50%, 60% of the heatmap of fingerprint 105.

According to some exemplary embodiments, Note the data is also filtered as follows before computing the TF map.

The data was also filtered as such: filtering between 0.075 Hz and 70 Hz & notch filtered of 33 Hz:

According to some exemplary embodiments, the heatmap of fingerprint 105 represents an EEG feature space.

According to some exemplary embodiments, to prepare the EEG feature space, the EEG time series is represented in the time-frequency domain. In some embodiments, the log-power of eight frequency bands is extracted from the time series of each channel using, for example the Matlab function bandpower. In some embodiments, the bandpower estimation is performed in sliding windows of 1 [sec] and an overlap of 0.5 [sec], resulting in a time course with a sampling rate of 2 Hz. (The resulting time series representing the power in each frequency band were further submitted to a spike removal procedure). In some embodiments, to account for the hemodynamic response in the fMRI data, time delayed versions of each feature in steps of 0.5 [sec] up to 30 [sec] is added, hence generating 60 shifted time series per band and channel. In some embodiments, the resulting time were further normalized into z-scores, leaving each frequency with a mean of zero.

In some embodiments, this feature extraction step results in a multidimensional normalized feature space which is defined as follows [Channels×Frequency bands×Delays×TimeSamples]/[CH*FQ*D*T].

According to some exemplary embodiments, the fingerprint 105, for example an EEG space is generated as described in Hasson et al. “One-Class FMRI-Inspired EEG Model for Self-Regulation Training”, Plos One 2016.

In an example, a fingerprint is stored as a representation (in memory) of a set of coefficients for a regression matrix which estimates a fMRI signal, for example a fMRI-BOLD signal of one or more specific brain regions. Each coefficient of the set of coefficients is a coefficient of a specific feature comprising specific power bands/frequencies, one or more electrodes and one or more time delay windows. For example, a VS fingerprint as shown in FIG. 1b includes a set of coefficients, with each coefficient representing a specific one of 8 electrodes, a specific one of 8 frequency bands and a specific time window of 30 time windows.

In the experiment, 25 fingerprints were used, and the results of the 25 fingerprints were summed. In some embodiments, the VS fingerprint is generated by combining multiple signatures, for example by at least one of, averaging, voting, throwing out outlier or any other statistical method used for combining two or more sets of numerical values, to generate a single VS fingerprint.

As described above, each coefficient model includes a specific number of frequency band, where each frequency band is defined by an upper limit frequency value and a lower frequency value. In a VS fingerprint according to some embodiments of the invention, the range of frequencies of each power band may vary, for example, an upper limit and/or a lower limit, by about 5%, 10%, 20%, 25% or intermediate percentages. Such varying may be non-uniform. For example, more variance may be allowed for lower frequencies than higher frequencies. For example, Specifically, a lower or higher frequency value of a power band of a frequency below 20 Hz may vary in up to 50% between different VS fingerprints. Optionally or additionally, in frequencies above 20, for example, varying of the frequency may be allowed up to 25%.

In addition, each fingerprint model generates a prediction of BOLD at a time delay after the EEG data. For example, for VS, a time delay of 30 seconds is used as part of the model. It is believed that this may represent the hemodynamic response shown in fMRI-BOLD activation of a brain region. This hemodynamic response of the brain region results in a time difference between measured fMRI data indicating an activation of the brain region and EEG data that correlates with the brain region activation. Other delays may be used as well, for example, optionally within a range of 25%. In some embodiments, a shorter delay may be used, for example, 50% shorter.

The time delay of 30 seconds is divided into several overlapping time windows, optionally each one a second long. A length of each time window within the time delay of 30 seconds and a degree of overlapping between time windows may vary, for example, in a range of 50%-200%, for other VS fingerprints.

For example as shown in FIG. 1b, the VS fingerprint includes regions with intensity values in the highest quartile of intensity levels, for example in:

Power band 3, within a time delay window between −4 and −8 seconds for electrode C4;

Power band 3, within a time delay window between −5 and −7 seconds for electrode F8 and for electrode T8;

Power band 4, within a time delay window between −4 and −7 seconds for electrode P8;

Power band 5 within a time delay window between −5 and −8 seconds for all electrodes;

Power band 6 within a time delay window between −5 and −8 seconds for electrodes C4, F7, F8, T8, P8 and TP10;

Power band 7, within a time delay window between −5 and −8 seconds for electrodes T8 and P8.

For example as shown in FIG. 1b, the VS fingerprint includes regions with intensity values in the lowest quartile of intensity levels, for example in:

Power band 1, within a time delay window between −2 and −5 seconds for electrode F7;

Power band 3, within a time delay window between −10 and −14 seconds for electrodes C4 and F7;

Power band 4, within a time delay window between −10 and −14 seconds for all electrodes;

Power band 5, within a time delay window between −11 and −13 seconds for electrodes F7;

Power band 6, within a time delay window between −12 and −14 seconds for electrodes F8;

Power band 6, within a time delay window between −11 and −14 seconds for electrodes TP9.

A fingerprint may include one or more of the regions with intensity values in the lowest quartile of intensity levels, and/or one or more regions with intensity values in the highest quartile of intensity levels, for predicting an activity, for example fMRI-BOLD, of the VS with lower accuracy.

### VS Fingerprint Generation

In one method of fingerprint generation, fMRI and EEG signals are recorded from participants, while the participants selectively activate the VS, for example by voluntary or involuntary responding to a signal or a stimulus. The recordings of the signals form a plurality of datasets. In an experiment, fMRI and EEG signals were recorded from 14 subjects while listening to selected musical compositions. Each of the participating subjects selected 5 neutral musical compositions that did not trigger an emotional feeling in the subject, and 5 favorable musical compositions that trigger a positive feeling (pleasure) in the subject. This numbers are not essential for generating a VS signature and optionally serve to give statistical diversity. Listening to the different types of musical compositions allowed selective activation of the VS, which is optionally used for generating a localizer (for the VS). All together 2×15 minutes of signals per subject were recorded, to generate 25 data sets, due to corruption of some recorded signals.

In some embodiments, the data sets generated by the recordings are subjected for cross-validation, for example a “leave one out” validation process. In the experiment, 25 “leave one out” cross validation processes were performed. In each round of the cross validation processes, 24 of the 25 datasets were used to generate the model and one dataset to test it, where each of the datasets already includes selected frequency power bands 1 to 8, a selected number (30) of time delay windows (1 second long), as noted above.

In some embodiments, regression is applied on each of the data sets, for example to select a number of electrodes which represent the data. Then, the data for the selected electrodes is used to build coefficients for a regression matrix. In the experiment, group partial least square (PLS) regression was applied on each data set of the 25 data sets to select electrodes, resulting in a selection of 8 electrodes, though other methods may be applied as well. This resulted in a selection of 8 electrodes, C4, F7, F8, T7, T8, P8, TP9, and TP10.

In some embodiments and in the experiment, the matrix was tested using the “leave one out” cross validation. In the experiment, the 3^{rd} component of the PLS regression was used. In some embodiments, when the results of the cross-validation are good results, PLS can be performed on all the datasets, for example the 25 data sets of the experiment without performing the “leave one out” cross validation.

In the experiment, the data processing resulted in a VS signature that includes 25 matrices. These 25 matrices are applied in the processing of newly measured EEG signals, and then the results combined, for example by at least one of averaging, outlier rejection, voting, etc. In other embodiments, the signatures are first combined into a single matrix which is then applied to a stream of acquired EEG data.

Below is an example for how to use the VS fingerprint to predict an activation level of the VS:

Record or receive 8 EEG streams, one per a single EEG electrode attached to a head of a subject. The EEG electrode is attached to the head of the subject at positions C4, F7, F8, T7, T8, P8, TP9, and TP10.

For each stream perform a time-frequency decomposition to extract relevant frequency bands. Each frequency band is defined by a range of frequencies. For example, FFT is used to extract power at each of a set of the frequency bands. Then, assign an intensity power at each time window for each frequency and for each electrode (of the 8 electrodes), resulting in a matrix which includes 8×8×30 values.

The data is obtained within a time delay window of 30 seconds. The time delay window optionally reflects the hemodynamic response in the fMRI data, resulting in a time difference between a fMRI-BOLD signal, and EEG signals correlating with the fMRI-BOLD signal. Duration of the time delay window between a fMRI-BOLD signal, and EEG signals correlating with the fMRI-BOLD signal depends on a brain region, for example a duration of a time delay window for the Amygdala can be set to be about 15 seconds, whereas the time delay window of the VS has a duration of 30 seconds.—

Then, multiply the obtained data with the above fingerprint, sum it together and add an intercept value to obtain an activation value representing a predicted fMRI-BOLD activation of the VS.

In some embodiments of the invention, the obtained value is treated as a relative value, for example a value that is relative to a baseline value. As noted herein, baseline values may be obtained, for example, by a baseline session (e.g., neutral-enjoyment music). Alternatively, the obtained relative value allows to monitor changes in activity over time, changes in activity in a specific subject, changes in activity between different situations and/or between different stimuli. One example is using the output as a neuro-feedback signal, which may be used to assist a patient in relative changes in VS activity.

Below is an example of the VS fingerprint, arranged by electrodes. Each comma separated lien reflects a power band, from the bands 1 to 8, as shown in FIG. 1b. An optional intercept coefficient is provided at the end:

Electrode 1
, 0.0032508, 0.0013711, 0.0015937, 0.0018316, 0.0020579, 0.0021995, 0.0023128, 0.0022573, 0.0020198,
0.0016218, 0.0012903, 0.0012532, 0.0012822, 0.0011754, 0.00086328, 0.00058774, 0.00041185,
0.000040076, −0.00057319, −0.0009794, −0.00055851,
0.00066874, 0.0021913, 0.0033724, 0.0035681, 0.0028038, 0.0015487, 0.00036675,
0.000048757, 0.00060016
, 0.0012301, 0.00067077, 0.000093305, 0.000058644, 0.00034685, 0.00045444, 0.00022619, −0.000048413,
0.000048207, 0.00028573, 0.00039251, 0.00037231, 0.00017304, −0.0001359,
−0.00043704, −0.00062194, −0.00075119, −0.0010069, −0.0012291, −0.0010565, −0.00025639,
0.00095611, 0.0018579, 0.0019315, 0.0010119, −0.00072467, −0.0022303, −0.0024356, −0.0011699,
0.0011977
, 0.0032393, 0.0030041, 0.0023267, 0.001527, 0.0007776, 0.00039264, 0.00057963, 0.001097, 0.0015312,
0.00153, 0.0012088, 0.00085199, 0.00066679, 0.00061643, 0.00048912, 0.00026848,
−0.00014134, −0.00070248, −0.0013542, −0.0020259, −0.0024479, −0.0023708, −0.0014664,
−0.00043373, −0.0001575, −0.00069195, −0.0015146, −0.0016006, −0.0010148, 0.00029114
, −0.00024397, −0.00014636, −0.000039397, −0.00025493, −0.00022669, −0.000011598,
0.00016718, 0.00014525, −0.000022093, 0.000020707, 0.000056058, −0.00018672,
−0.00078081, −0.001594, −0.0022253, −0.002666, −0.003251, −0.0037348, −0.0041401, −0.0039953,
−0.0025653, −0.00046183, 0.0016755, 0.002908, 0.0027649, 0.001647, 0.00014521, −0.00055845, −0.00045143,
0.00043825
, −0.00054842, −0.00085839, −0.00060833, −0.00040412, −0.00013358, −0.000044504, −0.000239,
−0.00069292, −0.0012789, −0.0014653, −0.0013459, −0.0013168, −0.0016907, −0.0025083,
−0.0034062, −0.0040503, −0.0044472, −0.0047947, −0.0052065, −0.0051403, −0.0039361, −0.0016433,
0.00098993, 0.0029304, 0.0036969, 0.0032572, 0.0019924, 0.0006747, −0.000020488,
0.00030086
, 0.000011233, −0.000038702, 0.000082345, −0.00016764, −0.00044029, −0.00052468, −0.00053338,
−0.00049667, −0.00035497, 0.000039851, 0.00053415, 0.00097272, 0.0011532, 0.00069469,
−0.00028681, −0.0012945, −0.0021057, −0.002836, −0.0032802, −0.0025002, −0.00033424,
0.0023717, 0.0046309, 0.0056308, 0.0054438, 0.0044531, 0.0031865, 0.0023663, 0.0021114,
0.0023899
, −0.00057852, −0.00040605, 0.00032262, 0.00055009, 0.00055466, 0.00040854, −0.000019292,
−0.00034161, −0.00049155, −0.00051507, −0.0005213, −0.00048739, −0.00060937, −0.0008765,
−0.0011923, −0.0018793, −0.0028682, −0.0038538, −0.0041436, −0.0029458, −0.00044684,
0.0026358, 0.0051351, 0.0060493, 0.0056612, 0.0043023, 0.0022867, 0.00070257, −0.000050864,
0.0004295
, 0.00073473, 0.00095296, 0.0013197, 0.0014675, 0.001465, 0.0015291, 0.001672, 0.001813, 0.0015993,
0.00097095, 0.00063616, 0.00068733, 0.00072356, 0.00064063, 0.00028771, −0.00024195,
−0.00077675, −0.0011816, −0.0012875, −0.00089114,
0.00016095, 0.0017563, 0.0032562, 0.0037164, 0.0028816, 0.0011816, −0.00055647,
−0.0015404, −0.0017486, −0.0012237
Electrode 2
, 0.00070758, 0.00049338, −0.00025269, −0.0009255, −0.0013884, −0.0017095, −0.001858,
−0.0018608, −0.0015341, −0.0012014, −0.00087436, −0.0006717, −0.00087913, −0.0011346,
−0.0013718, −0.0014764, −0.001551, −0.0017398, −0.0017797, −0.0014721, −0.00073918, −0.00003815,
0.00034971, 0.00026222, −0.00067257, −0.0020628, −0.0029786, −0.0026502, −0.0011273,
0.0010057
, 0.00066419, 0.000018634, −0.00018295, −0.00015185, −0.000060117, −0.000052758,
0.00021234, 0.00088702, 0.0017318, 0.002494, 0.002735, 0.0024054, 0.0016548, 0.00076575,
0.00017968, −0.00011634, −0.00039218, −0.00072495, −0.0011188, −0.0014491,
−0.0015202, −0.0010496, 0.000037281, 0.00093638, 0.00091939, −0.000087826, −0.0012008,
−0.0013355, −0.00088714, −0.000012145
, −0.0019864, −0.0017544, −0.0013893, −0.0012296, −0.0010716, −0.0009679, −0.00090243,
−0.00088089, −0.00090242, −0.00061405, −0.00011588, 0.000026799, −0.00043221, −0.0012362,
−0.0021782, −0.0033317, −0.0044603, −0.0049082, −0.0046576, −0.0036195, −0.0016275,
0.0010505, 0.0038132, 0.0052698, 0.005004, 0.0034412, 0.001404, 0.00025895, −0.000023561,
0.00038129
, −0.0027354, −0.0027693, −0.0022269, −0.0015309, −0.00081375, −0.0005802, −0.0010266,
−0.0018673, −0.0025876, −0.002662, −0.0021926, −0.0017535, −0.0018274, −0.0025201, −0.0035602,
−0.0045315, −0.0054826, −0.0063584, −0.0069843, −0.0067703, −0.0049794, −0.0019106,
0.0013967, 0.003555, 0.0039315, 0.0027576, 0.00098557, −0.00017214, −0.000655, −0.00055504
, −0.0023021, −0.0017432, −0.00069458,
0.000094315, 0.00044102, 0.00064851, 0.00066921, 0.00049209, 0.00029133, 0.00021964,
0.0004141, 0.00069477, 0.00078391, 0.00053649, −0.00030855, −0.0013917, −0.0019911,
−0.0021474, −0.0020444, −0.0013134,
0.00040931, 0.0030921, 0.0057528, 0.0068418, 0.0059714, 0.0038593, 0.0015832, 0.00027649,
0.00023972, 0.0012295
, −0.0023606, −0.0022679, −0.0016685, −0.00097686, −0.00026799, 0.00032157, 0.00036519,
−0.00012285, −0.00065721, −0.00064851, −0.000058859, 0.00033208, −0.000031177, −0.00085342,
−0.0016588, −0.0023815, −0.0029017, −0.0031193, −0.0030955, −0.002258, −0.00025327,
0.0025489, 0.0053579, 0.0068266, 0.00654, 0.0050258, 0.0028942, 0.0013709, 0.00083114,
0.0011522
, 0.00015621, 0.000036679, 0.00026436, 0.00039132, 0.00050502, 0.00074741, 0.00089267, 0.0010284,
0.0010751, 0.00099081, 0.0010534, 0.0011325, 0.00087939, 0.00022909, −0.00045659,
−0.0010978, −0.0017567, −0.0021934, −0.0022454, −0.0015555, −0.00025111,
0.0013641, 0.003025, 0.0038934, 0.0036631, 0.0027147, 0.0015822, 0.00080908, 0.00059024,
0.00096467
, −0.00049432, −0.00060245, −0.00042828, −0.00017677, −0.000020081,
0.00024463, 0.00029495, 0.00025871, 0.00051874, 0.00085073, 0.0011718, 0.0011349,
0.00063869, 0.000088394, −0.00037329, −0.00067877, −0.0010568, −0.0016263, −0.0019706,
−0.0017403, −0.0010727, −0.00046784, −0.000096738, −0.000058898, −0.00039082, −0.00090462,
−0.0014601, −0.0015981, −0.0013011, −0.00055526
Electrode 3
, 0.0010437, 0.00067, 0.00028431, 0.00031537, 0.00063499, 0.00095976, 0.0012803, 0.0016046, 0.0020599,
0.0023537, 0.0022101, 0.0018706, 0.001394, 0.00085239, 0.00015065, −0.00077904,
−0.001616, −0.0020814, −0.0020135, −0.0015488, −0.00077586, −0.000006805, 0.00027054,
−0.00025973, −0.0018563, −0.0038506, −0.0049521, −0.0044527, −0.0027281, −0.00058738
, 0.0007168, 0.00044882, 0.00012396, −0.00018603, −0.00029759, −0.00024115, −0.000089511,
0.0002235, 0.00064819, 0.0010912, 0.0015802, 0.001796, 0.0015705, 0.0011318, 0.00056888,
0.000092018, −0.00035396, −0.00091382, −0.0012228, −0.0010317, −0.00045952,
0.00025194, 0.00082919, 0.00087767, 0.00016148, −0.0010904, −0.0020506,
−0.0018409, −0.00074135, 0.00082015
, −0.0002613, 0.00010998, 0.000031542, −0.00078997, −0.0015317, −0.0018226, −0.0017137,
−0.001426, −0.0013442, −0.0012341, −0.0010709, −0.0010689, −0.0014072, −0.0023463, −0.0033976,
−0.0042542, −0.0049355, −0.0051816, −0.0051364, −0.0043864, −0.0026527, −0.00048477,
0.0016142, 0.0028432, 0.0027758, 0.0017343, 0.00053856, 0.000034962, 0.00017413,
0.00062256
, −0.00021689, −0.000065307, −0.00031933, −0.00088265, −0.0012559, −0.0013766, −0.0012865,
−0.001185, −0.0011309, −0.0010306, −0.001088, −0.0011458, −0.0013444, −0.0020476, −0.0029153,
−0.0039512, −0.0051877, −0.0062849, −0.0068406, −0.0062639, −0.0042459, −0.0011037,
0.0017517, 0.0031334, 0.0027291, 0.00094018, −0.00080433, −0.0017096, −0.0016768, −0.0007263
, 0.0015304, 0.0014579, 0.0015823, 0.0017047, 0.0017581, 0.0014986, 0.00099941, 0.00049243, 0.00019992,
0.0001549, 0.00014873, 0.00031209, 0.00035886, −0.00029618, −0.0015817, −0.0030096,
−0.0039505, −0.0042642, −0.0042569, −0.0034434, −0.0014891,
0.001192, 0.0038701, 0.0051124, 0.0044268, 0.0026064, 0.00059587, −0.0005645,
−0.00074594, −0.00013741
, 0.00012782, −0.00024187, −0.000479, −0.00031101,
0.00015961, 0.0010943, 0.0020646, 0.0023919, 0.0022261, 0.0019346, 0.001652, 0.001511,
0.0013003, 0.00076457, 0.000070106, −0.00089024, −0.002055, −0.0030577, −0.0033828, −0.0022042,
0.00056326, 0.0040999, 0.0070274, 0.0078774, 0.0063196, 0.0033294, 0.00044318,
−0.0012364, −0.0014083, −0.00049313
, −0.00034172, −0.00041624, −0.00033987, −0.00030735, 4.3813E−06,
0.00078204, 0.0018144, 0.0024875, 0.0026634, 0.0024034, 0.0016563, 0.00067492, −0.0002229,
−0.00075003, −0.00088864, −0.0007482, −0.00064922, −0.00084776, −0.0010368, −0.00060544,
0.0006529, 0.0023933, 0.0036655, 0.0035209, 0.0019245, −0.00044281, −0.0022876,
−0.0028061, −0.0021985, −0.001011
, 0.0013953, 0.0025712, 0.0029288, 0.0024772, 0.0017492, 0.00090807, 0.00026513, 0.000010255, 0.00017945,
0.00067656, 0.00088853, 0.00058551, 0.000052757, −0.00052556, −0.00093584,
−0.0012738, −0.0017498, −0.0022157, −0.0024538, −0.0021314, −0.0011378,
0.00029536, 0.0015006, 0.0016537, 0.00074097, −0.00064487, −0.0016808, −0.0016114, −0.00051797,
0.00099725
Electrode 4
, −0.00053781, −0.00073994, −0.0011344, −0.0012832, −0.0011825, −0.0011841, −0.0011436,
−0.001134, −0.00095854, −0.00044915,
0.00019412, 0.00072953, 0.00093198, 0.00096957, 0.00077041, 0.00018961,
−0.00064134, −0.0012485, −0.0011904, −
0.0005822, 0.00021815, 0.000839, 0.0012134, 0.0010905, 0.00015389, −0.0012775, −0.0022929,
−0.0020574, −0.0010584, 0.00025233
, 0.0003265, 0.000053176, −0.00017694, −0.00037045, −0.0003797, −0.00033966, −1.7655E−06,
0.00071081, 0.0014525, 0.0020623, 0.0022624, 0.0019249, 0.0013461, 0.00072128, 0.000047115,
−0.00060318, −0.0013506, −0.0020424, −0.0023991, −0.0024183, −0.002067, −0.0013344, −0.00012631,
0.0010156, 0.0012721, 0.00045366, −0.00084777, −0.0016264, −0.0017495, −0.0010897
, −0.0012459, −0.0012728, −0.0010165, −0.00091803, −0.00064663, −0.00026032, −0.000008467,
−0.000016974, −0.00030879, −0.00048884, −0.00042112, −0.0003534, −0.00050398, −0.00088521,
−0.0013926, −0.0021681, −0.0033301, −0.0043002, −0.0047933, −0.0045471, −0.0030222, −0.00052632,
0.0022284, 0.0039026, 0.0037622, 0.0024575, 0.00083207, 0.00011116, 0.00023797, 0.00089483
, −0.0020381, −0.002433, −0.0025586, −0.0024128, −0.0018692, −0.0012145, −0.0007121, −0.000471,
−0.00047935, −0.00061902, −0.00097389, −0.0015067, −0.0023589, −0.0034725, −0.0043286,
−0.0047675, −0.0050644, −0.0054398, −0.005825, −0.0056945, −0.0042536, −0.0016409,
0.0010414, 0.0027301, 0.0028394, 0.0014346, −0.00058853, −0.0018893, −0.0019938, −0.0011465
, −0.0019591, −0.0016829, −0.0011464, −0.00084028, −0.00079438, −0.00090912, −0.0010319,
−0.00098288, −0.00065109, −0.00014393, 0.00046566, 0.00087716, 0.00063901, −0.00035308,
−0.001699, −0.0026781, −0.0033316, −0.003706, −0.0034624, −0.0020574,
0.00067286, 0.0037717, 0.0062353, 0.0070486, 0.0059119, 0.003712, 0.0015047, 0.00039104,
0.00045844, 0.0012453
, −0.0010082, −0.00074887, −0.00035866, −0.00031374, −0.00052849, −0.00062692, −0.00037406,
−0.00016962, −0.0002014, −0.00041248, −0.00064848, −0.00056354, −0.00053385, −0.00096692,
−0.0017772, −0.0028033, −0.003639, −0.0038899, −0.0035073, −0.002015,
0.00077781, 0.0041763, 0.0069576, 0.007761, 0.0063834, 0.0034655, 0.00033022,
−0.0014303, −0.0015057, −0.00042894
, −0.0009628, −0.00095007, −0.00049549, −0.000030022,
0.00042662, 0.0010407, 0.0018261, 0.0024077, 0.0024624, 0.0021843, 0.0020325, 0.0019398,
0.0014751, 0.00071384, 0.000074403, −0.00040506, −0.0010041, −0.0017637, −0.0024599,
−0.0025445, −0.001773, −0.00015553, 0.0015667, 0.0021197, 0.0013057, −0.00014612, −0.0011709,
−0.0014162, −0.0011039, −0.00037768
, 0.00036004, 0.000056131, −0.00019375, −0.0003496, −0.00020979,
0.00021525, 0.00067057, 0.00093286, 0.0010855, 0.0011676, 0.0010189, 0.00044409,
−0.00063923, −0.0015799, −0.0018253, −0.0015321, −0.0013506, −0.0018133, −0.0023878,
−0.0025109, −0.0023012, −0.0019659, −0.0016699, −0.0015446, −0.0018012, −0.0024326, −0.0028991,
−0.0028102, −0.0022649, −0.0013408
Electrode 5
, 0.00041893, 0.000087192, −0.00033619, −0.00040906,
0.000085229, 0.00069855, 0.0010769, 0.0013215, 0.0015381, 0.0015938, 0.0014038, 0.00095794,
0.00010228, −0.00077933, −0.0013192, −0.0017119, −0.0019473, −0.0020268,
−0.0020278, −0.0016256, −0.00056972, 0.00067867, 0.0014918, 0.0012744, −0.00028808,
−0.0023766, −0.0035719, −0.0032107, −0.0016831, 0.00024433
, 0.0011892, 0.00047678, −0.00028634, −0.00083463, −0.00074159, −0.00031671,
0.000025832, 0.00040581, 0.00079836, 0.00096389, 0.0010209, 0.00092159, 0.00056332,
0.00015147, −0.00029539, −0.00051178, −0.00051765, −0.00069629, −0.00080028, −0.00053911,
2.9816E−06, 0.00066315, 0.0012776, 0.0013823, 0.00066388, −0.00057157,
−0.0016705, −0.0019196, −0.0012978, −0.00012689
, −0.000021627, 0.00040026, 0.00046537, −0.00011082, −0.00066809, −0.00088125, −0.0009405,
−0.001017, −0.0012341, −0.0012735, −0.0010494, −0.00084497, −0.00076746, −0.0011846,
−0.0019404, −0.0026691, −0.0033958, −0.0038694, −0.0040742, −0.0035956, −0.0022127, −0.00048925,
0.0011823, 0.0022666, 0.0025484, 0.0021103, 0.0012432, 0.00064273, 0.00042469, 0.00053862
, 0.00044603, 0.00065288, 0.00063477, 0.00026018, 0.00012712, 0.000053816, −0.00013259,
−0.00063188, −0.0013968, −0.0018699, −0.0019522, −0.0014943, −0.0010967, −0.0015234,
−0.0026359, −0.0042637, −0.0057262, −0.0065948, −0.0069109, −0.0061917, −0.0042808, −0.0015646,
0.0008618, 0.002104, 0.0020534, 0.00074658, −0.00092346, −0.0019945, −0.0021351, −0.0011262
, −0.00025052, −0.0001836, −0.000082707, −0.00030486, −0.00027754, 1.7204E−06, 0.00011082,
−0.00010849, −0.00047237, −0.00053604, −0.00019041, 0.00042961, 0.00083858, 0.00056479,
−0.000333, −0.0014163, −0.0020623, −0.0021775, −0.0022136, −0.0017054, −0.0001127,
0.002163, 0.0043456, 0.0052232, 0.0046002, 0.0031287, 0.0014284, 0.00047678, 0.00023159,
0.0005134
, −0.0021002, −0.0026368, −0.0026645, −0.0023327, −0.0016638, −0.00085465, −0.00014594,
0.000162, 0.0002773, 0.00036612, 0.00022804, 0.000049815, −0.00045685,
−0.0012543, −0.0019182, −0.0025283, −0.0030217, −0.003502, −0.0036661, −0.002715, −0.00067008,
0.0018054, 0.0036969, 0.0041893, 0.003233, 0.0011865, −0.0011132, −0.0026258,
−0.002888, −0.00192
0.00011921, 0.00025686, 0.00035585, 0.00018447, 0.00032235, 0.00097751, 0.001862, 0.0025451,
0.0027845, 0.0026431, 0.0021614, 0.0012338, 0.00026664, −0.00027625, −0.00067604, −0.0010728,
−0.0014715, −0.0019866, −0.0024172, −0.0023887, −0.0016269, −0.000059698,
0.0016123, 0.0022073, 0.001334, −0.00048645, −0.0022007, −0.0028813, −0.0024726, −0.001511
, 0.0013369, 0.0024006, 0.0028099, 0.0025281, 0.0020616, 0.0015291, 0.00087742, 0.0003973, 0.00044686,
0.0010683, 0.0018366, 0.0019753, 0.0013896, 0.00044913, −0.0002399, −0.00050063,
−0.00084965, −0.00117, −0.0013703, −0.0011215, −0.0002518, 0.00072629, 0.0013513, 0.0010008,
−0.00016902, −0.0014846, −0.0024512, −0.0023262, −0.0011251, 0.00055947
Electrode 6
, 0.0010642, 0.00084056, 0.00038686, 0.00027106, 0.00023181, −0.000042098, −0.00043715,
−0.00080944, −0.0007242, −0.00044255, −0.0002896, −0.00028022, −0.00045825, −0.00060486,
−0.00076485, −0.0010233, −0.0014138, −0.0018346, −0.0018832, −0.0013693, −0.00042897,
0.00064785, 0.0015205, 0.001881, 0.0013856, −0.000056033, −0.0015868, −0.0020568, −0.0012595,
0.00055204
, 0.0016902, 0.0013806, 0.00092953, 0.00049993, 0.000067134, −0.00015441,
0.00019284, 0.00092014, 0.0016202, 0.0019181, 0.0017338, 0.0012726, 0.00077486, 0.00038535,
0.000072991, −0.00018954, −0.00054696, −0.00088386, −0.0010813, −0.0012548,
−0.0012918, −0.0010103, −0.00017927, 0.00066368, 0.00075266, 0.000014047, −0.00099438,
−0.0013691, −0.0012215, −0.00043167
, −0.00099186, −0.0010168, −0.00076884, −0.0006996, −0.00046807, −0.00019154, −0.00007343,
−0.00015396, −0.00037944, −0.00034128, −0.00008385, 7.1662E−06, −0.00028123, −0.00086894,
−0.0014925, −0.0021629, −0.0029635, −0.0034145, −0.0035238, −0.0030686, −0.0015789,
0.00052388, 0.0028015, 0.00407, 0.003722, 0.0023494, 0.00070623, 0.000035826, 0.00014348,
0.00066905
, −0.00037736, −0.00059935, −0.00041203, −0.0002621, −0.000023123, 0.000060633, −0.00016251,
−0.00070322, −0.0014317, −0.0018893, −0.0020447, −0.002135, −0.0024999, −0.0033374, −0.0043642,
−0.0051674, −0.0057728, −0.0062726, −0.0066312, −0.0063555, −0.0047773, −0.0021234,
0.00079729, 0.0030009, 0.0038341, 0.0031771, 0.0015196, 0.000076293, −0.00049508, −0.00014531
, −0.0022614, −0.0022232, −0.0018395, −0.0016547, −0.0017009, −0.0017957, −0.0017285, −0.001444,
−0.00093859, −0.00039367, −0.000014101, 0.00017922, 0.000067917, −0.00053056, −0.0016268,
−0.0025911, −0.0031624, −0.0035922, −0.003677, −0.0027658, −0.00056105,
0.0024387, 0.0050788, 0.0060822, 0.0054582, 0.0040336, 0.0026057, 0.0019035, 0.0018175,
0.0021573
, −0.0021536, −0.0024091, −0.0022299, −0.0021883, −0.0019731, −0.0015124, −0.0011594,
−0.0010007, −0.0011174, −0.0014414, −0.0016848, −0.0015929, −0.0013977, −0.0013576, −0.001478,
−0.0019238, −0.0027466, −0.0036556, −0.0040069, −0.0029913, −0.00041389,
0.0031628, 0.0064362, 0.0080487, 0.0077121, 0.0056544, 0.0028828, 0.0010322, 0.00052762,
0.0011856
, −0.0010657, −0.0010135, −0.00057739, −0.00016461,
0.00023223, 0.00063952, 0.00086266, 0.0010439, 0.0010819, 0.00094744, 0.0011085,
0.0013094, 0.0010802, 0.00057537, −0.000021725, −0.00063126, −0.0012073, −0.0016448,
−0.0016638, −0.0010788, −
0.000070154, 0.0013916, 0.003153, 0.0041993, 0.0039301, 0.0025946, 0.00084908, −0.00041288,
−0.00088816, −0.00039882
, −0.00069512, −0.00099976, −0.00090095, −0.00075972, −0.0005364, −0.000042718,
0.00037924, 0.00071824, 0.0010072, 0.0011501, 0.001262, 0.0011076, 0.00044354,
−0.00037034, −0.00069565, −0.00037797, −0.000012048, −0.000079893, −0.0002962, −0.000071339,
0.00055653, 0.0010533, 0.0013363, 0.0013809, 0.00097247, 0.00018496,
−0.0007053, −0.0011108, −0.00073359, 0.00019358
Electrode 7
, 0.0015083, 0.0012229, 0.00074521, 0.00049276, 0.00039884, 0.00024463, −0.000092941,
−0.00033774, −0.00022993, −0.000073224, 0.000031171, 0.000019158, −0.000048296,
0.000080279, 0.00028529, 0.00054479, 0.00065457, 0.00022703, −0.00037214, −0.00051188,
0.00012045, 0.0013426, 0.0023616, 0.0024335, 0.0011525, −0.0010242, −0.0024861,
−0.0023234, −0.00067505, 0.0019197
, 0.0025214, 0.0023041, 0.0017437, 0.0010049, 0.0001955, −0.00029341, −0.0001431,
0.00031517, 0.00054693, 0.00049122, 0.00034271, 0.00010665, −0.000061749, −0.00010154,
0.000032302, 0.00030099, 0.00017544, −0.00042008, −0.001279, −0.0021111,
−0.0025018, −0.0023091, −0.0014606, −0.00064141, −0.00059641, −0.0013814, −0.0022737,
−0.0022183, −0.0013351, 0.00013658
, 0.000065186, 0.00017761, 0.00012113, −0.00031851, −0.00040358, −0.00010589,
0.00021005, 0.00028817, 0.000084823, 0.000071051, 0.00019281, 0.000051511,
−0.00054177, −0.0015319, −0.0023886, −0.0029368, −0.0034477, −0.0037537, −0.0040382,
−0.0038235, −0.0024721, −0.00054315, 0.0014585, 0.0024966, 0.002128, 0.00084058, −0.00076452,
−0.0015761, −0.0013773, −0.00014724
, −0.001121, −0.001269, −0.00064157, −0.000096928, 0.00018207, 0.000014107, −0.00065514,
−0.0015366, −0.0023491, −0.0026444, −0.002547, −0.0024572, −0.0025748, −0.0031777, −0.0041303,
−0.0049475, −0.0054903, −0.0058645, −0.0061441, −0.0056449, −0.0037774, −0.00090632,
0.0022209, 0.004367, 0.005048, 0.0046817, 0.0035908, 0.0025262, 0.0019352, 0.0018777
, −0.00047404, −0.00035548, −0.00027446, −0.00082161, −0.0014168, −0.0015987, −0.0014365,
−0.0010615, −0.00059902, −0.00013259, 0.00016012, 0.00032713, 0.0004298, 0.00021507,
−0.00039083, −0.0011026, −0.0017471, −0.0023324, −0.0026096, −0.0018524,
0.00015033, 0.002824, 0.0051615, 0.0061115, 0.0057334, 0.0045902, 0.0032819, 0.0026061,
0.0025057, 0.0028801
, −0.0012572, −0.0016862, −0.0012326, −0.0007696, −0.00043581, −0.00033546, −0.00068731,
−0.0010977, −0.0013901, −0.0014307, −0.0012844, −0.001266, −0.0014925, −0.0018537, −0.0020565,
−0.0022104, −0.0024894, −0.0028701, −0.0030225, −0.0021238, −0.00010977,
0.0024763, 0.0048336, 0.0059931, 0.0060746, 0.0051837, 0.0033604, 0.0017361, 0.00054866,
0.00032066
, −0.0011789, −0.00097954, −0.00037347,
0.000040986, 0.00039393, 0.00088342, 0.0012812, 0.0015281, 0.0013332, 0.00085013
, 0.000804, 0.0010601, 0.0011329, 0.0008638, 0.00025219, −0.00053714, −0.0012194, −0.0014071, −0.00093123,
0.00023709, 0.0017723, 0.0033529, 0.0046898, 0.0049716, 0.003953, 0.0020494,
−0.000026452, −0.0013241, −0.0017177, −0.0012388
, −0.00041107, −0.00017286,
0.00057806, 0.0012239, 0.0015642, 0.0018028, 0.0017653, 0.0015145, 0.0011441, 0.00077839,
0.00081358, 0.00084373, 0.00037821, −0.00028533, −0.00072342, −0.00063599,
−0.00032843, −0.00033087, −0.00040528, −0.000011052,
0.00083377, 0.0016325, 0.0020568, 0.0019311, 0.0011895, 0.00018649,
−0.00068916, −0.00096849, −0.0006032, 0.00009488
Electrode 8
, −0.000038801, −0.00042577, −0.00073712, −0.00069522, −0.00025079, 0.000095194, 6.9143E−06, −0.000057266,
0.0002174, 0.00062698, 0.00075129, 0.00048819, −0.00015536, −0.0009014,
−0.001284, −0.0015206, −0.0017767, −0.0019946, −0.0019984, −0.0013287,
0.000091393, 0.0015921, 0.0025449, 0.0025208, 0.0011924, −0.00082855, −0.0023666,
−0.0026434, −0.0018038, −0.00016066
, 0.0013037, 0.00097207, 0.00053949, 0.00016194, 0.00021802, 0.00047577, 0.0006354, 0.00079097,
0.00096711, 0.001036, 0.0010861, 0.0011058, 0.00091206, 0.00048481, −0.00010097,
−0.00044126, −0.00046107, −0.00053946, −
0.00047883, 0.00010393, 0.0011234, 0.0024129, 0.0035123, 0.0036142, 0.0024653, 0.00056501,
−0.0010062, −0.0014567, −0.00086637, 0.00030819
, −0.0002501, −0.00012812, 0.000011483, −0.00025911, −0.0005352, −0.00057286, −0.00067337,
−0.00088032, −0.0011126, −0.0011853, −0.00099372, −0.00076223, −0.00066863, −0.0010023,
−0.0016783, −0.0024247, −0.0031015, −0.00353, −0.0039128, −0.0038461, −0.0029663, −0.0014762,
0.00027362, 0.0015286, 0.0019671, 0.0015141, 0.00049337, −0.0002073, −0.00025915,
0.00020412
, −0.00013331, 8.6474E−06,
0.00034874, 0.00047288, 0.00062381, 0.00064829, 0.00047625, 0.00004874, −0.00035857,
−0.00037193, −0.000193, 0.00023529, 0.00036356, −0.00022028, −0.0013645, −0.0030462,
−0.0044681, −0.0052656, −0.0056879, −0.0052447, −0.0036234, −0.0010357,
0.0016432, 0.0031448, 0.0029809, 0.0014433, −0.00045993, −0.0015865, −0.001467, −0.00015038
, −0.000093839, 0.00014214, 0.0003863, 0.000077505, −0.00017544, −0.00024836, −0.0003485,
−0.00062455, −0.00088805, −0.00064176, −0.00014092, 0.00039621, 0.00071403, 0.00047717,
−0.00011493, −0.00080745, −0.00138, −0.0017595, −0.0020657, −0.0017254, −0.00023692,
0.0019226, 0.0038799, 0.0047294, 0.0044452, 0.0033583, 0.0018676, 0.00081997, 0.0002282,
0.00035127
, −0.0015998, −0.0022408, −0.002582, −0.0025404, −0.0021521, −0.0017047, −0.0012723, −0.0011345,
−0.0010908, −0.00095003, −0.0010285, −0.0012762, −0.0018166, −0.0025022, −0.0030936,
−0.0036314, −0.004019, −0.0044219, −0.0046231, −0.0038908, −0.0020033,
0.00067999, 0.0030755, 0.0039075, 0.0030215, 0.00093249, −0.0014168, −0.0026126,
−0.0023521, −0.00093514
, 0.00047561, −0.00020851, −0.00086267, −0.0016347, −0.0019669, −0.0014337, −0.00021323,
0.0010117, 0.0016183, 0.0013954, 0.00056989, −0.00043336, −0.00132, −0.0017901,
−0.0019182, −0.0018963, −0.0019579, −0.0022977, −0.0025719, −0.0022948, −0.0011745,
0.00059032, 0.0021182, 0.0025491, 0.0016279, −0.00018848, −0.0019956, −0.0029722,
−0.0028087, −0.0018983
0.00012285, 0.00067463, 0.0013645, 0.0017515, 0.001921, 0.0018827, 0.0016764, 0.0013208, 0.00086421,
0.00053301, 0.00037478, 0.00036506, 0.00043606, 0.00049223, 0.00053122, 0.00044908,
0.00010044, −0.00065552, −0.0014903, −0.001734, −0.0012749, −0.0005539, −0.0001449, −0.00040159,
−0.0012091, −0.0022886, −0.0033608, −0.0037048, −0.003008, −0.001406
Intercept 0.0016374

### Exemplary Monitoring Activity of a Mesolimbic System Brain Region

According to some exemplary embodiments, activity of at least one specific brain region of the mesolimbic system is monitored using recorded electrical signals, for example EEG signals. In some embodiments, the activity of the at least one specific brain region is monitored without a need for spatial scan data, for example fMRI data. In some embodiments, the function of the mesolimbic system and/or the function of the reward system is estimated, for example to determine if a subject suffers from a difficulty in self-modulating of the reward system. Optionally, estimating the function of the mesolimbic system and/or the function of the reward system allows, for example to diagnose a subject with a reward system-related disease, for example with apathy and/or anhedonia.

Reference is now made to FIG. 1C, depicting a process for monitoring an activity of at least one brain region of the mesolimbic system and/or at least one brain region of the reward system, according to some exemplary embodiments of the invention.

According to some exemplary embodiments, at least one stimulus is provided to a subject, at block 128. In some embodiments, the at least one stimulus is selected to affect an activation level of at least one specific brain region of the mesolimbic system. Alternatively or additionally, the at least one stimulus is selected to affect an activation level of at least one specific brain region of the reward system. In some embodiments, the stimulus is selected based on an ability of the stimulus to promote engagement of the subject with the stimulus, for example in a way that modifies the activation of the at least one specific brain region.

According to some exemplary embodiments, the stimulus comprises an audio and/or a visual stimulus, for example in a form of music and/or a movie. In some embodiments, the stimulus is provided to the subject by at least one of a display, a speaker, headphones and earphones.

According to some exemplary embodiments, an activity of at least one specific brain region of the mesolimbic system is determined at block 130. In some embodiments, the activity of the at least one specific brain region is determined based on electrical signals recorded from the subject brain, for example EEG electrical signals. In some embodiments, the electrical signals are recorded by one or more electrodes attached to a head of the subject, for example to a scalp of the subject. Optionally, the electrical signals are recorded during the providing of the stimulus.

According to some exemplary embodiments, the activity of the at least one specific brain region is determined by identifying a correlation between at least a portion of the recorded electrical signals and an activation fingerprint of the at least one specific brain region indicating, for example an activity level of the at least one specific brain region. Optionally, the activation fingerprint indicates a specific fMRI-B OLD activation of the at least one specific brain region. Alternatively, the activation fingerprint indicates a change in activation of the at least one specific brain region.

According to some exemplary embodiments, a subject is diagnosed with a reward system-related disease if an activity level of the at least one specific brain region is not changed in response to the stimulus, at block 134. In some embodiments, the reward system-related disease comprises anhedonia and/or apathy. In some embodiments, the subject is diagnosed with the disease, if the activity of the at least one specific brain region remains within a range of up to 10%, for example up to 5%, up to 3%, up to 1% or any intermediate, smaller or larger percentage value, following the providing of the stimulus compared to a baseline activity level. Optionally, the baseline activity level was determined prior to providing the stimulus at block 128.

According to some exemplary embodiments, if an activity level of the brain region is increased, the stimulus is modified at block 136. In some embodiments, the stimulus is modified in a way that promotes a positive feedback loop in activation of the at least one specific brain region, for example in a healthy subject. Optionally, the stimulus quality is increased according to the increase in the activity of the at least one specific brain region. In some embodiments, increasing a quality of a stimulus comprises increasing a harmony of the stimulus, or reducing a degradation level of the stimulus. Optionally, the electrical signals are recorded from the subject brain while modifying the activity of the brain region.

According to some exemplary embodiments, the subject is diagnosed with the reward system-related disease if an increase in activity of the at least one specific brain region following the providing of the modified stimulus is smaller than a target increase level, for example if the increase is smaller than 10%, smaller than 5%, smaller than 3%, smaller than 1% or any intermediate, smaller or larger percentage value, compared to a previously determined activity level of the at least one specific brain region. Optionally, the previously determined activity level of the specific brain region is determined prior to the providing of the modified stimulus to the subject.

According to some exemplary embodiments, a subject diagnosed with the reward system related disease is optionally treated with a neurofeedback treatment, at block 138. Alternatively, the subject is treated with a neurofeedback treatment in combination with at least one drug.

### Exemplary Delivery of a Positive Feedback

Reference is now made to FIG. 1D, depicting a process for providing a positive feedback signal to a subject selected to increase an activation of at least one specific brain region, according to some exemplary embodiments of the invention.

According to some exemplary embodiments, electrical signals, for example EEG electrical signals are recorded from a deeply located brain region, at block 142. In some embodiments, the deeply located brain region is a brain region located underneath a cortex of the subject. Optionally, the deeply located brain region is a brain region having a lower activity level compared to an activity level of the deeply located brain region in a healthy human subject.

According to some exemplary embodiments, during the recording of the electrical signals, the subject is optionally instructed to perform one or more tasks and/or to apply one or more strategies. Optionally, the tasks and/or strategies are selected based on an ability to increase an activation of the deeply located brain region, directly, or indirectly, for example by increasing an activity of a brain region associated with the deeply located brain region.

According to some exemplary embodiments, an increase in activation of the at least one specific brain region is identified at block 144. In some embodiments, the increase is identified by identifying a relation between at least a portion of the recorded electrical signals and an electrical fingerprint, for example an EFP, of the deeply located brain region indicating at least one of activation of the deeply located brain region, a specific activation level of the deeply located brain region, and/or a change in activation of the deeply located brain region.

According to some exemplary embodiments, a positive feedback signal is provided to the subject at block 146. In some embodiments, the positive feedback signal is provided with parameter values selected to promote a positive feedback loop is the activation of the deeply located brain region in the subject. In some embodiments, the positive feedback signal is provided and/or the parameter values are determined according to the identified increase in the activation of the brain region. In some embodiments, the positive feedback signal comprises an audio signal and/or a visual signal. In some embodiments, the parameter of the feedback signal comprise at least one of quality, volume, harmony, and duration of the feedback signal.

### Exemplary Improving a Quality of a Degraded Feedback Signal

According to some exemplary embodiments, a degraded feedback signal is provided to a subject, as part of a neurofeedback process, for example a neurofeedback treatment procedure or a neurofeedback training procedure. In some embodiments, during the neurofeedback process, the degraded feedback signal is improved, according to an activity level of a specific deeply located brain region, for example a specific brain region located underneath the cortex. Reference is now made to FIG. 1E, depicting an improvement of a neurofeedback signal, according to an increase in activation level of a specific brain region, according to some exemplary embodiments of the invention.

According to some exemplary embodiments, a feedback signal, for example an audio signal and/or a visual signal is degraded at block 152. In some embodiments, in case the feedback signal is an audio signal, for example a musical composition, the musical composition is degraded compared to a previous and optionally familiar version of the musical composition. In some embodiments, the musical composition is degraded by modifying, for example replacing one or more musical notes with a different musical note, or by switching an order of one or more musical notes of the musical composition. Alternatively or additionally, the musical composition is degraded by modifying a volume, for example sound level of the musical composition, pitch, flow and/or speed of the musical composition.

In some embodiments, in case the feedback signal is a visual signal, for example a movie, the movie is degraded compared to a previous and optionally familiar version of the movie. In some embodiments, the movie is degraded by removing and/or replacing one or more pixels, changing the speed and/or the volume of the movie.

According to some exemplary embodiments, the degraded feedback signal is delivered to the subject at block 154. In some embodiments, the degraded signal is delivered by an interface, for example a patient interface comprising at least one of a display, a speaker, headphones and/or earphones.

According to some exemplary embodiments, electrical signals are recorded from a deeply located brain region, at block 156. In some embodiments, the electrical signals, for example EEG electrical signals are recorded by one or more electrodes attached to the head of the subject, for example to the skull of the subject. Optionally, the electrical signals are recorded. In some embodiments, the electrical signals are recorded as previously described at block 142 in FIG. 1D.

According to some exemplary embodiments, during the recording of the electrical signals, the subject is optionally instructed to perform one or more tasks and/or to apply one or more strategies. Optionally, the tasks and/or strategies are selected based on an ability to increase an activation of the deeply located brain region, directly, or indirectly, for example by increasing an activity of a brain region associated with the deeply located brain region.

According to some exemplary embodiments, an increase in activation of the deeply located brain region is identified at block 158. In some embodiments, the increase in activation is identified using the electrical signals recorded at block 156, and for example as previously described at block 144 of FIG. 1D.

According to some exemplary embodiments, a quality of the feedback signal is increased, for example improved, at block 160. Additionally, the improved feedback signal is delivered to the subject, optionally, while recording the electrical signals at block 156. In some embodiments, the quality of the feedback signal is improved, for example by modifying the feedback signal, to be more similar to a previously and more familiar version of the feedback signal. In some embodiments, the quality for the feedback signal is improved, for example by removing at least some of the degrading modifications introduced when the feedback signal is degraded at block 152.

### Exemplary Detailed Process for Generation of a VS Fingerprint

Without being bound by any theory or mechanism of action, the assignment of reward value is an important driving force of human behavior. A large body of evidence has pointed to the important role of ascending mesolimbic dopamine signaling in forming a core reward system. Converging evidence suggests that major nodes in this mesolimbic pathway such as the ventral striatum (VS), Ventral Tegmental Area (VTA) and ventromedial prefrontal cortex (vMPFC) are involved in processing diverse types of incentives such as food and money, and recent evidence indicates that this system is also engaged by musical stimuli. These studies further highlighted local dopamine release as one of the correlates of reward processing. Correspondingly, reward circuit disturbances have been associated with symptoms of anhedonia and apathy—some sources of distress in various psychiatric disorders. Yet, treatment of these symptoms to date is limited. Therefore, there is a growing need for non-invasive accessible methods that selectively monitor and target in real-time the ascending mesolimbic system with ease of accessibility.

According to some exemplary embodiments, electrical signals, for example EEG electrical signals, and scan data, for example fMRI data are received from one or more subjects, for example 2, 5, 10, 20, 30 or any intermediate, smaller or larger number of subjects. In some embodiments, the EEG electrical signals and the fMRI data are recorded simultaneously. Optionally, the EEG electrical signals and the fMRI data are recorded while the one or more subjects performs at least one activity that modulates an activity level of the VS. In some embodiments, the at least one activity comprises a reward-related task and/or a task that activates the mesolimbic system. In some embodiments, a task that activates the mesolimbic system comprises a pleasurable naturalistic music listening task, a monetary incentive delay (MID), adoor guessing task, a gambling task, a Punishment, Reward, and Incentive Motivation (PRIMO) game, Safe or risky domino choice task (Kahn et al., 2002), viewing of highly pleasing pictures or video clips, listening to highly pleasing sounds, reminiscence of positive memories or any modification thereof. Alternatively or additionally, the at least one activity comprises pharmacological manipulation, for example administration of a dopaminergic agonist.

In some embodiments and in an experimental process performed to generate the VS-EFP, structural and functional scans were performed using a 3T Siemens MAGNETOM Prisma scanner (Siemens, Erlangen, Germany) with a 20-channel head coil. Functional whole-brain scans were performed in an interleaved top-to-bottom order, using a T2*-weighted gradient-echo echo-planar imaging sequence (TR/TE=2620/30 ms, flip angle=90°, 64×64 matrix, FOV=192×192 mm, 43 slices per volume with 3 mm thickness and no gap). Positioning of the image planes was performed on scout images acquired in the sagittal plane. A total of 345 volumes were acquired for each of the music listening sessions and between 278 and 332 for the MID sessions. 3D anatomical T1-weighted imaging was obtained using MPRAGE sequences with 1 mm iso-voxel to provide high-resolution structural images.

In some embodiments and in an experimental process performed to generate the VS-EFP, EEG data were recorded concurrently with the fMRI scan. The data were acquired using a battery operated MR-compatible BrainAmp-MR EEG amplifier (Brain Products, Munich, Germany) and the BrainCap electrode cap with sintered Ag/AgCl ring electrodes providing 30 EEG channels and 1 electrocardiogram (ECG) channel (Falk Minow Services, Herrsching-Breitbrunn, Germany). The electrodes were positioned according to the 10/20 system with a frontocentral reference. The signal was amplified and sampled at 5 kHz and was further recorded using the Brain Vision Recorder software (Brain Products, GmbH, Gilching, Germany).

### Data Analysis and Preprocessing

Step 1—fMRI and EEG Preprocessing:

In some embodiments and in an experimental process performed to generate the VS-EFP, the recorded fMRI data and the received EEG signals were preprocessed. In some embodiments and in the experiment, the fMRI preprocessing, which was done, for example, using Brain-voyager QX (Brain Innovation, Maastricht, The Netherlands), optionally included at least one of slice timing correction, motion correction using sinc interpolation and high-pass filtering of 3 cycles per scan. In some embodiments and in the experimental process, each functional data-set was then manually co-reregistered to the corresponding anatomical map and incorporated into a 3D dataset via, for example, trilinear interpolation. In some embodiments and in the experimental process, the obtained data was then transformed into Talairach space and was optionally spatially smoothed using a Gaussian kernel (isotropic 4-mm FWHM).

In some embodiments and in the experimental process, pre-processing of the EEG data, which was optionally done using the BrainVision Analyzer software (Brain Products, GmbH, Gilching, Germany), and included at least one of MR-gradient artifacts removal, down sampling to 250 Hz, band pass filtering between 0.075 Hz and 70 Hz, and Cardio-ballistic artifacts removal using semi automatic R peak detection. Additionally, the pre-processing further included a correction based on a subtraction of an averaged artifact template.

In some embodiments and in the experimental process, notch filtering of 33 Hz was applied, for example to account for a periodic noise of that frequency within the EEG data possibly due to scanner noise. Optionally, to account for possible artifacts due to head movements etc, an additional preprocessing step was applied for the detection of non-stationary components in the data using analytic approach for Stationary Subspace Analysis [SSA]. Using this approach, a component is considered ‘outlier’ if the associated eigenvalue is larger than a threshold: P₅₀+5·(P₇₅−P₂₅) where Pᵢ stands for the i^{th} percentile. Additionally, in each component, ‘problematic’ time period with significant higher than usual energy are detected and removed (by zeroing them out)].

Step 2—Defining a Target fMRI Signal and an EEG Feature Space for Predicting the Target fMRI Signal

In some embodiments and in the experimental process, the BOLD signal from bilateral VS was extracted by averaging over a map.

In some embodiments and in the experimental process, to extract an EEG model of VS activation related to reward processing, the BOLD signal from the VS (right & left) is extracted. Optionally, to ensure a functional relevance to reward processing, the VS region of interest (ROI) was defined using a Neurosynth map (www(dot)neurosynth(dot)org/), depicting a meta-analysis of the term reward. The ROI, was defined by applying a threshold of 14.5 to the forward inference meta-analysis map of “reward”. Time courses of BOLD activation were extracted for all voxels within this ROI mask and averaged across those voxels, such that for every run and participant, one time course was available. Then, to account for non-neural fluctuations within the extracted BOLD signal, the mean signal changes in white matter and cerebrospinal fluid were regressed out of the resulting time course using linear regression. The resulting BOLD signal was then up-sampled, for example to 2 Hz and normalized to z-scores (zero mean and one standard deviation).

In some embodiments and in the experimental process, to prepare the EEG feature space, the EEG time series in the time-frequency domain is represented, for example by extracting the log-power of eight frequency bands from the time series of each channel, optionally using the Matlab function bandpower.m. In some embodiments and in the experimental process, the band power estimation was performed in sliding windows, for example sliding windows of about 1 sec and an overlap of about 0.5 sec, optionally resulting in a time course with a sampling rate of about 2 Hz.

In some embodiments and in the experimental process, the division into bands followed division into the EEG frequency bands (in Hz) as follows: [0-2; 2-4; 4-8; 8-12; 12-16; 16-20; 20-25; 25-40]. Optionally, the resulting time series representing the power in each frequency band were further submitted to a spike removal procedure, whereby values exceeding a Median Absolute Deviation were replaced with the average signal.

In some embodiments and in the experimental process, to account for the hemodynamic response in the fMRI data, time delayed versions of each feature were added in steps of about 0.5 sec up to about 30 sec, for example to generate about 60 shifted time series per band and channel. Optionally or additionally, the resulting time were normalized into z-scores, leaving each frequency with a mean of zero.

In some embodiments and in the experimental process, the feature extraction step resulted in a multidimensional normalized feature space, for example an EEG signature or fingerprint which is defined as follows [Channels×Frequency bands×Delays×TimeSamples]/[CH*FQ*D*T]. This feature space was used to predict the BOLD activity in the VS, such that observed BOLD signal in time point T can be predicted from the EEG using the power of frequency bands FQ of a group electrodes CH in delays D from T.

### Step 3—“Fingerprinting”—Modeling of the Processed VS BOLD Signal Using the EEG Features Space

In some embodiments and in the experimental process, the model was trained in two main steps—during the first step, the channels to be used in the model were selected and during the second step, a partial least squares (PLS) regression was applied on the adjusted EEG feature space and fMRI data.

In some embodiments and in the experimental process, in each modeling iteration, the data entered to the model was the concatenated data of all the sessions. During the channel selection step, we modified the approach used in Witten D M et al., 2009 to fit a PLS model with a penalty on groups of coefficients (each group corresponds to a channel). The following optimization problem was solved:

$${max}_{{w}_{f} , {w}_{e}} {w}_{f}^{T} {\sum }_{fe} {w}_{e} ,$$

$$subject to {‖ {w}_{f} ‖}^{2} \leq 1 , {‖ {w}_{e} ‖}^{2} \leq 1 , {‖ {w}_{e} ‖}_{G} \leq c ,$$

where Σ_{fe} is the covariance matrix of the fMRI and EEG features time series, w_{e/f} are the weights whose aim is to maximize the covariance between the fMRI and EEG component, ∥⋅∥_{G} is the group lasso penalty and c is a parameter that controls the group lasso penalty. In case the fMRI data contains a single time series, we set w_{f}=1 and optimize wₑ. In our implementation, we are searching for the value of c such that the desired number of channels is selected.

In some embodiments and in the experimental process, following channels selection, the PLS model if fitted (matlab plsregress).

There are two parameters that need to be set, the number of selected channels (which we restricted to 8 for technical reasons) and the number of components of the PLS. Optionally, t estimate the best values for these parameters a grid search and the cross validation method were used. Values that maximize the average performance over the cross validation folds were chosen. As performance, the correlation between the model's output and the fMRI BOLD signal was used. Since there is much variability in the data and the noise patterns between the sessions, in order to correctly estimate the model's generalization capabilities we need to ensure that we train the model on data that is independent of the data we will use to estimate the performance. Hence, a leave-one-session-out cross-validation (LOOCV) method was used, where in each fold one of the sessions is left out and the model is trained on the rest.

In some embodiments and in the experimental process, to estimate the generalization error, an external LOOCV with an internal LOOCV was applied to decide the parameters. Optionally, for the final model fitting, we used LOOCV as before but considered only the sessions that performed well in the last step (correlation is higher than a threshold, in our case r_threshold=0.1). When applying the model on new data we average the predictions of the models we fitted in the CV.

### Step 4—Validation and Depiction of the Spatial Distribution of the Fingerprint

In some embodiments and in the experimental process, a common model coefficient matrix is generated, which is obtained by averaging the predictions of the models fitted in the cross validation. The model is then submitted to several complementary analysis lines that are designated to validate the model in additional contexts and depict the brain network configuration related to the extracted model of the VS.

Extraction of the predicted VS BOLD activity (i.e., VS-EFP): In some embodiments and in the experimental process the time-series of the VS-EFP was constructed by multiplying the recorded EEG data by the common model coefficient matrix. The EEG data (features) used for the model are a time/frequency matrices recorded from electrodes C4, F7, F8, T7, T8, P8, TP9 and TP10, including all frequency bands in a time window of 30 seconds.

The obtained VS-EFP was then submitted to a series of complementary validation analyses, which included the assessment of the EFP's: 1) Modeling performance: correlating between the VS-EFP and the NAcc-BOLD signal and assessing the statistical significance of the group's correlation coefficients; 2) Spatial specificity: highlighting of voxels that are strongly predicted by the VS-EFP. This was achieved by optionally applying a whole brain random effects general linear model analysis, with the VS-EFP as a regressor of interest; 3) Task related modulation: examining whether and how the VS-EFP is being modulated by reward similarly to the related tasks. This was achieved by applying a random effects general linear model analysis, with the VS-EFP as the dependent variable and the reward-related design (i.e., music-ratings) as the predictors.

### Statistical Analyses

fMRI Tasks Analysis

The statistical analyses were performed according to the random effects general linear model as implemented, for example in BrainVoyager QX software. The pleasurable and neutral conditions were modeled at two time scale; transient and sustained; the transient onset response to music was modeled as a 5 seconds long response time-locked to the onset of each excerpt; the sustained response was modeled as time-locked to 5 s after the onset of each excerpt, with a duration of 175 s. The reward-related responses to music were modeled based on the continuous ratings, which were provided following scanning, and were synchronized offline with the scan.

Responses were divided into moments of increase or decrease in rating as events time-locked to the moments in which participants pressed the button to provide an indication for a positive, or negative change in their rating, respectively per musical condition.

For the monetary incentive delay (MID) task, onsets of the anticipation, positive and negative feedback condition for the monetary or control trials were modeled time-locked to the moment in which the corresponding cue appeared. The response phase was further modeled time locked to the moment the moment in which the cue to perform the time estimation task appeared. In both tasks, the regressors were subsequently convolved with the canonical hemodynamic response function. Following model estimation, the difference between the increase and decrease in pleasure response was calculated to assess response to musical reward in the pleasurable music condition, and the difference between positive and negative feedback in the monetary conditions was calculated to assess the consummatory response to the monetary reward. The contrasts were submitted into a second level random effects analysis to assess the group effects.

VS-EFP BOLD correlates (EFP validation): A random-effects general linear model analysis was conducted according to the same principles described above, now using the VS-EFP time-series as the regressor of interest, and the contrast for this modulation was submitted to random effects analysis using a one sample t-test.

In the abovementioned general linear model analyses, six head motion parameters and mean signal in white matter were added to regress out motion and other non-neural related variance. Correction for multiple comparison was achieved by applying the Benjamini Hochberg procedure for controlling the false discovery rate (FDR). The statistical threshold of significance was further set with a minimal cluster-size of 4 contiguous functional voxels (>64 mm³).

VS-EFP Validation Analysis. VS-EFP Task-Related Modulation

To assess whether the VS-EFP is modulated by musical-pleasure, the VS-EFP signal was submitted to a two-level random effects general linear model analysis, using the same predictors that had used for delineating the BOLD response to the task (see above for details).

### Experiment 2: Feasibility of VS-EFP Modulation Via Musical-Neurofeedback:

Twenty participants were randomly assigned either to the VS-EFP-test (n=10) or to the EFP-yoked sham (n=10) group in a double-blind manner. Participants underwent a global rest block, followed by five neurofeedback (NF) blocks and one transfer block (with no feedback). The EFP-test group received continuous auditory feedback driven by their VS-EFP amplitude changes, calculated online every 3 seconds. The EFP-sham group received auditory feedback driven by the EFP of a participant from the VS-EFP group to whom he or she was “yoked”, hence unrelated to their own VS-EFP signal. In the first rest block, participants were instructed to clear their minds and rest with eyes closed and received no auditory feedback. In the subsequent five NF blocks, participants were presented with their self-selected musical pieces and were requested to ‘make the music sound louder’ by exerting mental strategies. No specific instructions regarding the desired strategy were provided. Each cycle included a passive listening baseline phase (‘attend’) and an active modulation NF phase (‘regulate’). During the ‘regulate’ phase, the music's volume was modulated in real-time, every 3 seconds, and in linear correspondence to the difference between the calculated VS-EFP in these two phases. To assess hedonic state and trait, participants filled out the Snaith-Hamilton Pleasure Scale (SHAPS), and the Positive and Negative Affect Schedule (PANAS).

Procedure: VS-EFP-NF Training. The VS-EFP-NF training consisted of one rest block, five NF blocks and one transfer block. In the first rest block, participants were given instructions to rest and received no auditory feedback. In the subsequent five NF blocks participants were instructed to passively listen to their self-selected music and rest for about 2:30 minutes (‘attend’, local baseline) and then, over a course of about 2 minutes, to make the music louder by exercising mental strategies (‘regulate’). The last transfer block was identical in its structure to the NF block, i.e., including an ‘attend’ and a ‘regulate’ phase, with the important exception that now participants were not presented with any music and received no feedback. A greater difference between the measured brain-activity in the ‘regulate’ vs ‘attend’ phases reflects better performance resulting in a higher sound volume. Instructions were intentionally unspecific, allowing individuals to adopt the mental strategy that they subjectively found most efficient.

Following each NF block, an experimenter entered the room and asked several questions about the NF experience (i.e., used strategies, subjective level of success and control, etc.). The VS-EFP group received continuous feedback driven by their own VS-EFP amplitude changes, calculated every 3 seconds. The EFP-sham control group, received auditory feedback based on the sham-yoked method, wherein each participant from the control group is paired to a participant from the test group, thus receiving the musical feedback of the paired test participant. This way, both groups were exposed to the exact proportion of sound manipulation that indicates their success-level, but only for the first group was it temporally related to VS activity. The experimenters and participants were blind to the group assignment, which was completely random for participants 2 to 19.

### Musical Feedback Generation.

The online EFP calculation and feedback generation was carried out via in-house Matlab scripts that were implemented an OpenViBE—an open source NF platform (Y. Renard et al., 2010). During the first Rest period, no feedback was generated. The Rest period was used to normalize each participant's VS-EFP, by using the mean and standard deviation across the VS-EFP value during rest. The auditory feedback consisted of five different self-selected musical excerpts, each presented in a different cycle. During the local baseline period, which lasted 2:30 minutes, the music played in a steady loudness level. During NF, volume changes were set in a linear scale, according to the real-time calculation of the VS-EFP. A predetermined change in VS-EFP value (either up or down) caused a respective change of 10 dB in the loudness of the music auditory feedback. After each NF period the SD was reset in accordance to the VS-EFP values recorded during the recent local baseline period.

### Preprocessing:

In some embodiments and in the experimental process, EFP data exceeding a value of 10 or 2.5 standard deviations from the mean of the entire signal was discarded. Cycles in which more than 20% of the data was discarded, were considered noisy and discarded from further analysis

### VS-EFP NF Statistical Analysis.

In some embodiments and in the experimental process, an index of VS-EFP amplitude upregulation was calculated as the difference between the ‘regulate’ and ‘attend’ phases [Mean (EFP-regulate)−Mean (EFP-attend)]. To test the hypothesis that the EFP group will show a significant increase in its VS-EFP amplitude during NF relative to baseline, student's t-test/Wilcoxon's sign rank test was applied per group, in comparison to the null hypothesis of zero upregulation. To test the hypothesis that the EFP-Test group would show a greater amplitude upregulation of VS-EFP during NF, a paired t-test/Wilcoxon's sign rank test was conducted using the mean VS-EFP upregulation a dependent variable, with sham-assignment as the pairing condition. As we had a-priori hypotheses regarding the direction of modulation, reported p values for this section are one-tailed. Finally, to assess the relevance of EFP-modulation to reward related indices, Spearman's correlations were calculated between VS-EFP upregulation and c-SHAPS scores, which represent individual differences in hedonic capacity (with lower scores indicating less anhedonia).

### Results

### Validation of Target Engagement.

To validate that the experimental context yielded the expected reward-related modulations within the VS, we first conducted an ROI analysis using the same bilateral VS mask that was used for the fingerprinting. As shown in FIG. 1F, random effects general liner model analysis within this ROI revealed an enhanced VS response to music at its onset, more so to the (self-selected) pleasurable than the (other-selected) neutral music.

A transient VS response to musical pleasure was further evident throughout listening. Specifically, while listening to pleasurable music there was an enhanced VS activation during moments of increase in pleasure ratings, which was greater than the response to moments of decrease in rating, as well as than the response to moments of increase in pleasure ratings while listening to the neutral music. Together these analyses demonstrated the functional relevance of the experimental design in engaging the target in mesolimbic reward circuit.

### Model Performance (VS-BOLD VS-EFP Correlation)

The graphs in the right panel of FIG. 1G depicts the frequency distribution of the coefficients of correlation between the time series of the VS-BOLD and the independently extracted VS-EFP model. In the modeling cohort, the mean correlation across runs was 0.206. Importantly, the correlation between the time series was further assessed in the independent replication datasets and was found to be significantly different from zero across all of the runs.

### Spatial Specificity

To highlight the brain regions that their BOLD activity correlated with the VS-EFP, a whole-brain random effects general liner model analysis was applied using the VS-EFP signal as a regressor of interest. As shown in FIG. 1G, the analysis revealed that the VS-EFP signal correlated with the VS-BOLD activity in the ROI that was used to develop the model both in the modeling cohort.

As shown in FIG. 1G, the VS-EFP in both datasets also consistently correlated with fMRI-BOLD activity of additional brain regions related to the mesolimbic network, including ventromedial prefrontal cortex (vMPFC), anterior midcingulate cortex (aMcc), anterior insula, as well as additional regions such as the Posterior Cingulate cortex.

### Generalization; EFP Signal Modulation in Different Reward-Related Context

We next tested if the association between VS-EFP signal and the VS-BOLD activity and its related network is evident under a different reward related context; the MID task. Correlation between the VS-EFP and VS-BOLD signal revealed that the correlation coefficients between the signals across the 20 participants were significantly different than zero (mean r=0.14). As shown in FIG. 1H, a whole-brain random effects general liner model analysis, with the VS-EFP as a regressor of interest further revealed that the VS-EFP signal correlated with the BOLD activity of the bilateral VS, albeit in a slightly more dorsal location than observed in the music task. Notably, the VS-EFP also correlated with activity in additional functionally relevant brain regions, including the VTA, and regions associated with the salience network such as the anterior insula, AmCC, as well additional regions such as the visual cortex, pre-SMA.

### VS-EFP Functional Relevance; Pleasurable Music Related Modulation

We next examined whether the VS-EFP is similarly modulated by musical reward as the VS-BOLD in the replication cohort. For that, we applied the same Random effects general liner model analysis, now with the VS-EFP, rather than the VS-BOLD, as the dependent variable and the various modeled responses to music as predictors. As shown in FIG. 1I, this analysis revealed a transient VS-EFP response to musical pleasure, as evidenced in the VS-BOLD. Specifically, there was an enhanced VS-EFP response at the onset of the pleasurable music, which was greater than the onset response to neutral music Enhanced EFP signal was also evident during pleasurable music listening, in moments of increase in pleasure ratings. Such response was greater than the response to moments of decrease in rating, as well as than the response to moments of increase in ratings during neutral music listening. It is notable that this response profile is similar to the VS-BOLD response in this cohort.

To assess this link, we examined the correlation between the selective responses mentioned above. Indeed, there was a positive correlation between the selective responses of the VS-EFP and VS-BOLD to pleasurable music; at its onset (i.e., onset pleasure>onset neutral; spearman correlation; r=0.72, p<0.01, one-tailed; and during moments of increased pleasure (rating increase>rating decrease; r=0.54, p<0.05; one-tailed).

### VS-EFP Application: Feasibility of Upregulation of the VS-EFP Using Neurofeedback

Finally, we set to explore whether the VS-EFP can be modulated within an NF context, and if such modulation ability further correlates with measures of anhedonia. For that, we conducted a sham-controlled study, in which participants were presented with their self-selected music and were requested to make the music sound louder. Changes in loudness were proportional to the relative change of the VS-EFP signal relatively to a local baseline period. In order to assess the modulation efficacy, we compared between the two groups' ability to regulate the VS-EFP across the five training cycles. Towards that goal, we calculated a VS-EFP modulation index as the difference between the NF and baseline phases [Mean (EFP signal)−Mean (baseline)], which was then averaged across the five cycles.

Analysis of VS-EFP modulation index indicated, as shown in FIG. 1J, that, as predicted, the EFP-test group succeeded to significantly modulate their VS-EFP activity (t(9)=4.005, p<0.005, one-tailed). Paired t-test further revealed a trend towards a group effect, whereby the test group (M=0.128, SE=0.032) better succeeded to upregulate their VS-EFP compared to the participants in the yoked-sham control group (M=0.066, SE=0.025; t(9)=1.812, p=0.052, one-tailed).

To further estimate the learning ability, we compared between the two groups' performance during the transfer cycle. This analysis revealed that the participants in the VS-EFP group (M=0.145, SE=0.042) were better able to upregulate their signal in the absence of any feedback, than the participants in sham group (M=0.051, SE=0.031; t(9)=2.39, p=0.02, one-tailed). Also consistent with our assumptions, VS-EFP modulation during training, as well as in the transfer trial negatively correlated with the levels of anhedonia among the test group (training: rₛ=−0.683, p<0.05; transfer: rₛ=−0.76, p<0.01, respectively), but not the control group (rₛ=−0.288, p=0.41; rₛ=−0.067, p=0.85, one-tailed). Fisher z test further revealed that the groups in fact differed in the strength of such negative association between anhedonia and NF-performance during the transfer trial (Fisher Z=−1.88, p=0.03, one-tailed).

In other words, participants who were more sensitive to reward (with lower levels of anhedonia scores), were more successful in learning to modulate their VS-EFP during VS-EFP training, and were also better able to generalize this ability to a transfer trial, when no feedback was provided.

### Exemplary Music Interface

According to some exemplary embodiments, a music interface is used to provide a feedback, for example a continuous feedback to a subject. In some embodiments, the music interface is used to provide a feedback to a subject with regard to an activation level of one or more brain regions, and/or one or more neuronal networks in the subject brain.

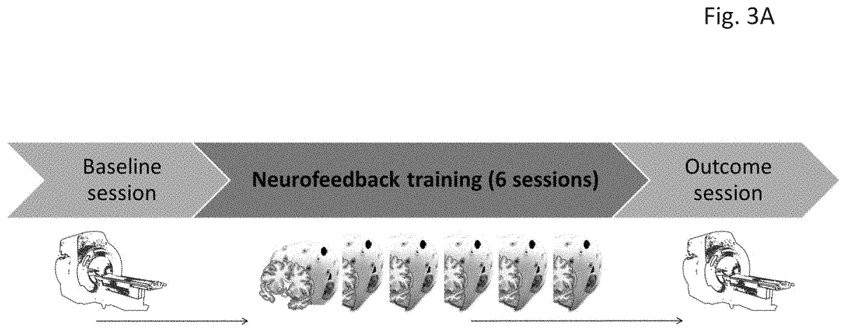

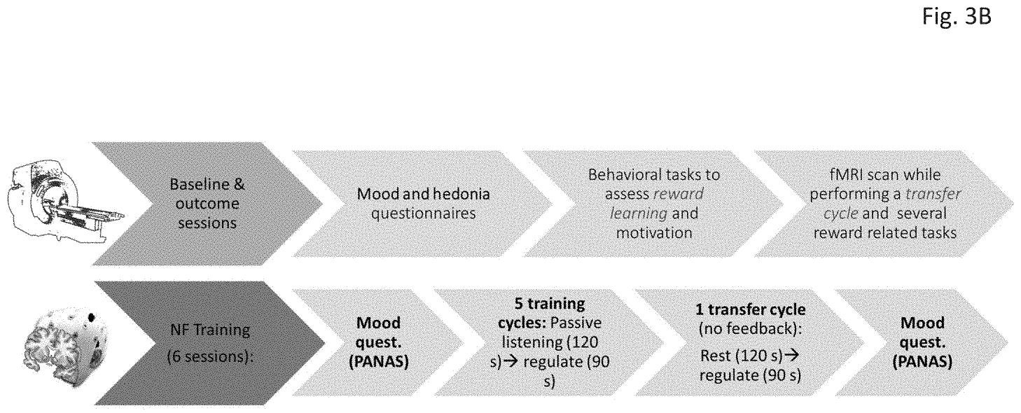

According to some exemplary embodiments, during a neurofeedback training, for example as performed in a validation study described in FIGS. 3A-6B, a trainee is presented with a self-selected musical piece and instructed to make the music increasingly pleasurable using a mental state. In some embodiments, the trainee is instructed to perform at least one motor task and/or at least one mental task that cause the music to sound more pleasurable to the subject. In some embodiments, on-line calculation of the user's VS-EFP signal modulation, for example in comparison to a local baseline affects the sound's quality, optionally via real-time application of acoustical distortion. In some embodiments, modulations are achieved by introducing one or more systematic manipulation to the audio spectrum. In some embodiments, the changes in sound's quality correspond with the extent of musical pleasure in a continuous fashion.

### Proof of Concept Validation Experiment

Musical pleasure is linked to recruitment of the ascending mesolimbic pathway, for example the ventral striatum (VS). Functional disturbances in this pathway are implicated in several devastating neuropsychiatric symptoms. As music has been shown to modulate the VS, an intriguing application is to harness music's power to determine if it is possible to train individuals to regulate their mesolimbic activity using neurofeedback.

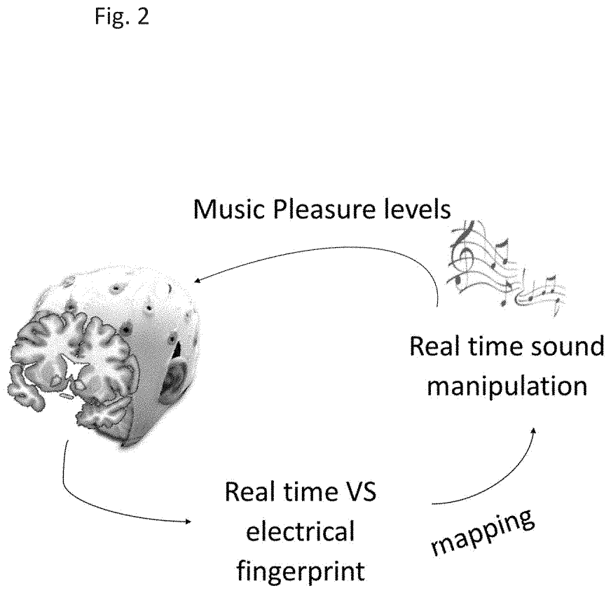

Neurofeedback is a training approach in which people learn to regulate their brain activity by using a feedback signal that reflects real-time brain signals. An effective utilization of this approach requires that the represented brain activity be measured with high specificity, yet in an accessible manner, enabling repeated sessions. In this validation experiment and according to some exemplary embodiments of the invention, a neurofeedback approach that utilizes an fMRI-inspired EEG model of mesolimbic activity, centered on the ventral striatum is used. In this neurofeedback approach a VS-electrical fingerprint (VS-EFP), for example as described in FIG. 1B, is combined with a pleasurable self-selected music interface, for example as shown in FIG. 2. In the validation study, the feasibility of this neurofeedback approach was tested by examining whether people can learn to regulate their VS-EFP signal with this music-interfaced approach. We hypothesized that repeated sessions of NF training to up regulate the VS-EFP with music would result in detectable changes in EFP-VS activity.

In the study, for example as shown in FIGS. 3A and 3B, twenty healthy participants (11 females, mean age 21.1+−2.77) underwent six neurofeedback training sessions, each consisting of five training cycles and one transfer cycle (with no feedback). The participants additionally underwent a pre- and post-assessment session that included fMRI scan, questionnaires and computerized tasks. In each training cycle and according to some exemplary embodiments of the invention, participants were presented with self-selected musical pieces and were requested to ‘make the music sound better’ by modulating their own brain activity.

The subjects in the study were divided randomly into a test group, where the subjects received musical feedback driven by changes in their own ventral striatum fingerprint (EFP), and into a control group where subject received musical feedback driven by changes in another participant's ventral striatum fingerprint.

In the experiment and in some embodiments, the music was modulated in real-time through an algorithm that introduced acoustical distortions that had been shown to affect musical pleasantness. In the experiment and in some embodiments, each cycle included a passive listening baseline phase (‘attend’) and an active modulation NF phase (‘regulate’). In the experiment and in some embodiments, a greater difference between the measured brain-activity in these two phases reflects better performance resulting in improved sound quality.

In the study, participants were randomly assigned to one of two conditions: Test group (n=10)—participants who received feedback driven by their own VS-EFP and a Control group (n=10), based on the sham-yoked method, wherein each participant from the control group is paired to a participant from the test group, thus receiving the musical feedback of the paired test participant. This way, both groups were exposed to the exact proportion of sound manipulation that indicates their success-level, but only for the first group was it temporally related to VS activity. VS-EFP power was calculated as the difference between the ‘regulate’ and ‘attend’ phases [Mean (EFP signal)−Mean (baseline)]. We then assessed learning improvement as the average difference between the best VS-EFP performance in training sessions two to six relatively to the first session average ([max(VS-EFP power session i)−max(VS-EFP power session 1]), where i stands for session number.

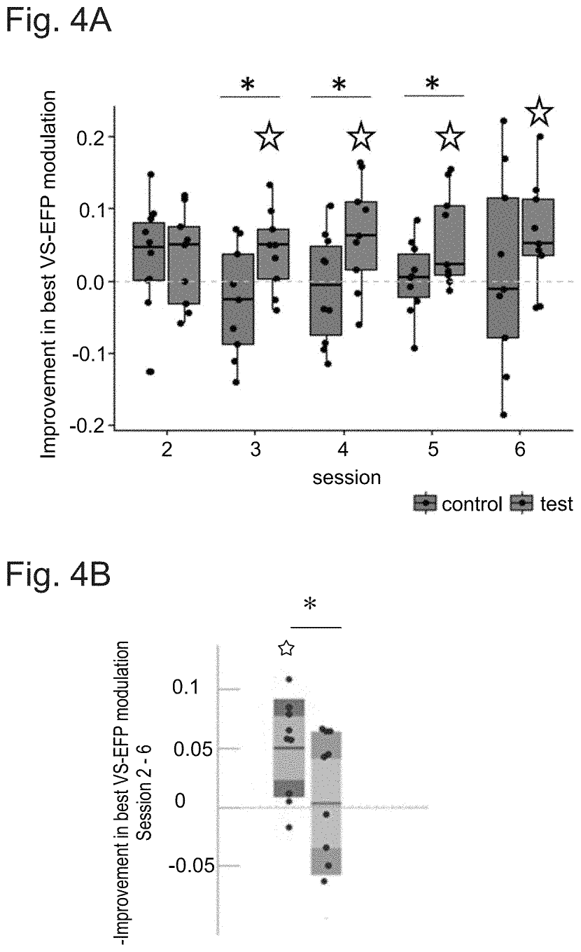

The study results demonstrated that the test group significantly improved in regulating their VS-EFP power relatively to the first session (Wilcoxon signed-ranks test, p<0.01, Z=42, one-sided), while the control group did not (p>0.077, Z=31). Importantly, such improvement was greater among test-group relatively to the yoked-sham group (Wilcoxon signed-ranks test for, p<0.01; Z=41; FIG. 4B).

The study findings provided compelling evidence that individuals are able to learn to self-regulate their mesolimbic activity via a music-interfaced Neurofeedback approach. In some embodiments, self-regulation of the mesolimbic activity, for example via a music-interfaced Neurofeedback approach, is used to treat apathy and anhedonia.

Reference is now made to FIGS. 3A and 3B depicting a design of the validation study. During the study, subjects repeated neurofeedback (NF) training with a pre NF training and post NF training neurobehavioral assessments. In pre NF training assessment session, the neurobehavioral assessment included a baseline session. In the post NF training assessment session, the neurobehavioral assessment included an outcome session. In the validation study, the NF training included 6 training sessions. In some embodiments, the NF training comprises at least one training session, for example 2, 3, 4, 5, 6, 7, 8 or any number of training sessions.

The baseline and outcome sessions performed during the study and in some embodiments of the invention included, answering mood and hedonia questionnaires, performing behavioral tasks, for example to asses reward learning and motivation, and performing an fMRI scan while performing a transfer cycle and several reward related tasks. Examples of behavioral tasks may include tasks assessing reinforcement learning (e.g., probabilistic selection task (MJ Frank etal., 2004), Probabilistic reward task (Pizzagalli, D. A etal., 2008), two-step decision task (Daw, N. D etal., (2011)), effort based decision making (e.g., effort expenditure for rewards task (Eefrt) (Treadway, M. T etal., 2009), gambling tasks, assessing music wanting and liking (Mas-herrero E. etal., 2018)

The NF training sessions performed during the study, and in some embodiments of the invention included filling a mood questionnaire (PANAS) at the beginning of the training sessions, 5 training cycles that included a passive listening stage (for 120 seconds) and a regulate stage (for 90 seconds). In the study and in some embodiments, during regulate the subjects perform one or more motoric or mental tasks to try and make the sound they hear more favorable and pleasant. In addition, the NF training sessions included performing a single transfer cycle, where no feedback is delivered to the subject. During this transfer cycle, the subjects are in rest for 120 seconds, and then apply the strategy they used to modulate the sound they hear during the training cycles, but without any feedback for up to 90 seconds. At the end of the training sessions, the subjects filled the mood questionnaire again.

Reference is now made to FIGS. 4A and 4B depicting changes in the VS fingerprint between different groups of the study.

With regard to FIG. 4A, the participants performed a neurofeedback training that included a rest stage and a regulate stage. The index of training performance: Improvement in best VS-EFP modulation: ([max(VS-EFP power session i)−max(VS-EFP power session 1]. VS-EFP power [Mean (EFP signal)−Mean (baseline)]. As shown in FIG. 4A, the test group showed a significant improvement in regulating their VS-EFP power starting the 3rd session (p<0.05 for all). The overall improvement across sessions was greater among the test-relatively to the control group (marginal main effect for group, F(1,16)=4.224; p=0.057).

FIG. 4B show group differences in VS-EFP signal modulation. The music-based VS-EFP-NF training led to a significant improvement in the VS-EFP-power upregulation among the test group but not the control group (denoted by a star). Importantly, such improvement in performance was greater for the test group than yoked-sham group (denoted by an asterisk).

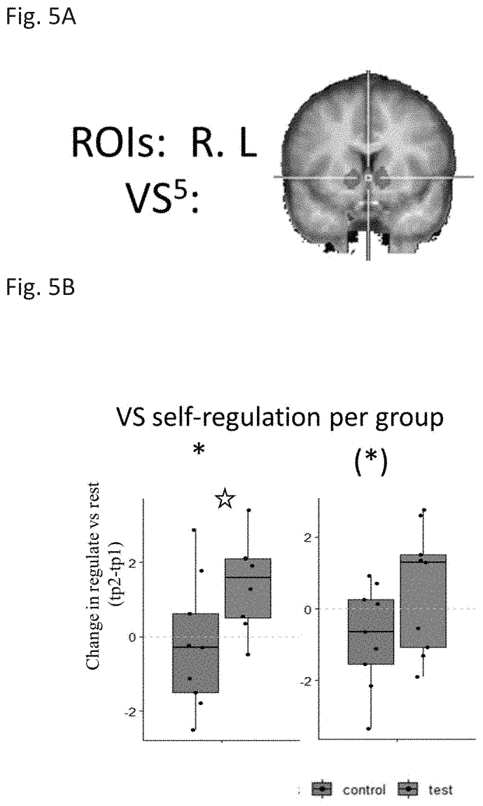

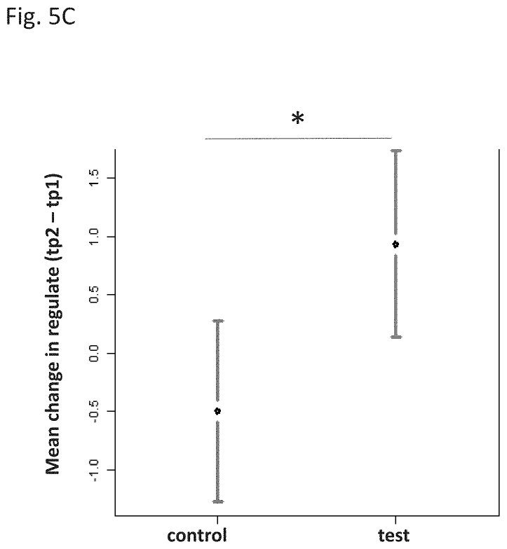

Reference is now made to FIGS. 5A and 5B, showing modulation of the ventral striatum activity, as measured with fMRI following NF training of the ventral striatum using the VS fingerprint.

During this analysis subjects performed an fMRI Task, which was similar to the transfer task in the NF training. This task included: rest stage (90 sec) followed a regulate stage (90 sec, no feedback), while being examined by fMRI.
The analysis included ROI based analysis in bilateral VS; Indexed performance as Change in VS regulation: [β regulate post)−[β regulate pre].

FIG. 5A show activation of the VS as shown in fMRI. The results shown in FIG. 5B demonstrate a positive and significant change in L. VS (left VS) regulation among the test group following training (t(7)=3.22 p<0.02). Such change was greater relatively to the control group ((t(14.33)=2.26, p<0.05).

In addition, FIG. 5C shows VS-BOLD self-regulation per group, the main effect for group across sides.

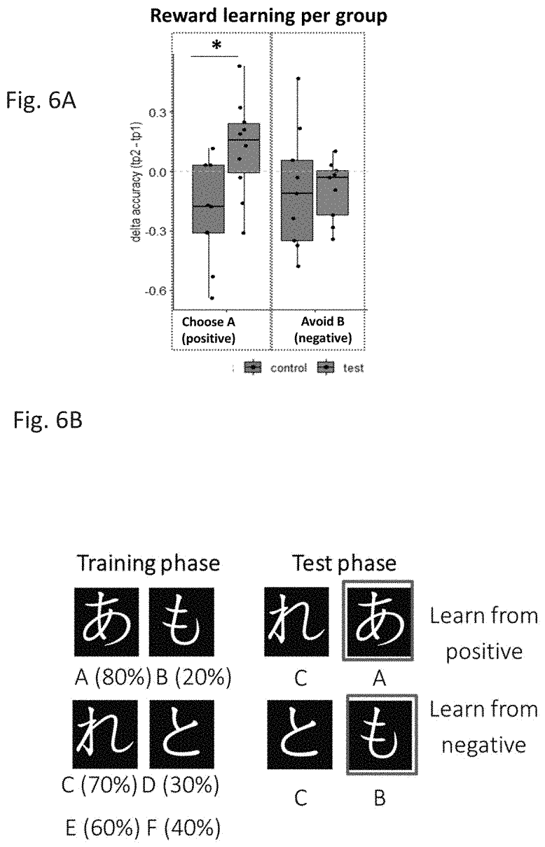

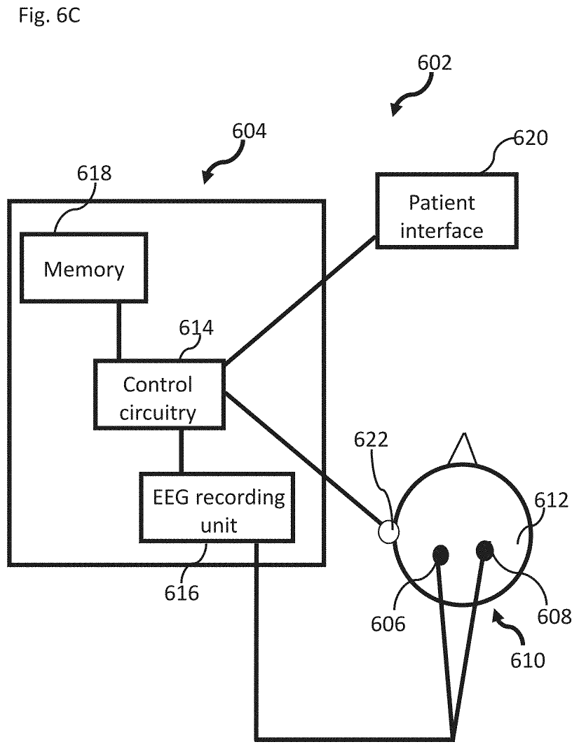

Reference is now made to FIGS. 6A and 6B showing an effect of the VS training using the VS fingerprint on reward-based learning.

The analysis included analyzing difference in accuracy between time points.

The analysis result showed that there was an interaction between the stimulus type and group (F(1,16)=4.73, p<0.05); Following training the test group exhibited a greater improvement in learning from positive rewards relatively to the control group (t(16.52)=2.91, p<0.01). No such difference was evident for learning to avoid negative outcomes.

### Exemplary System

According to some exemplary embodiments, a neurofeedback treatment is delivered by a system that collects information with regard to activation of one or more brain regions, for example one or more brain regions of the mesolimbic system, in a subject, and provides a feedback to the subject according to the activation of the one or more brain regions. Reference is now made to FIG. 6C, depicting a neurofeedback system, according to some exemplary embodiments of the invention.

According to some exemplary embodiments, a neurofeedback system, for example system 602, comprises a control unit 604 connectable to one or more electrodes, for example electrodes 606 and 608. In some embodiments, the one or more electrodes are part of the system. Alternatively, the one or more electrodes are commercially available electrodes, and the control unit is configured to be connected to the commercially available electrodes. In some embodiments, the electrodes 606 and 608 are attached to the body of a subject, for example patient 610.

According to some exemplary embodiments, the one or more electrodes, for example 606 and 608 comprise EEG electrodes attached to a head of the patient 610, for example to a skull of the patient 610. In some embodiments, the one or more electrodes are attached to the skull of the patient in one or more of the positions C4, F7, F8, T7, T8, P8, TP9 and TP10, derived for example from a 10-10 EEG system and/or from a 10-20 EEG system. Alternatively, the one or more electrodes are positioned at a distance of up to 10 cm, for example up to 5 cm, up to 3 cm or any intermediate, smaller or larger distance from at least one of the positions C4, F7, F8, T7, T8, P8, TP9 and TP10.

According to some exemplary embodiments, the control unit 604 comprises a control circuitry 614 connected to an EEG recording unit 616 of the control unit 604. In some embodiments, the EEG recording unit is connected to the one or more electrodes 606 and 608. In some embodiments, the control unit 604 comprises memory 618, for example a non-volatile memory. In some embodiments, the memory 618 stores at least one electrical fingerprint (EFP) of one or more specific regions of the mesolimbic system.

According to some exemplary embodiments, the stored at least one EFP correlates with an activation state of the one or more regions of the mesolimbic system. Alternatively or additionally, the stored at least one EFP is correlated with fMRI-B OLD activity of the one or more regions of the mesolimbic system. In some embodiments, the stored at least one EFP is an EFP of the Ventral Striatum (VS), indicating an activation state of the VS or a change in the activation state. In some embodiments, the stored at least one EFP correlates with fMRI-BOLD activity of the VS. Alternatively or additionally, the stored at least one EFP correlates with activity, for example fMRI-B OLD activity of at least one of ventromedial prefrontal cortex (vMPFC), anterior midcingulate cortex (aMcc), anterior insula, and the Posterior Cingulate cortex.

According to some exemplary embodiments, the memory 618 stores one or more algorithms, used for example for, processing electrical data, for example EEG data received from the one or more electrodes, identifying a relation between the EEG data and/or the processed EEG data, and the at least one stored EFP, and for detecting an activation level of one or more specific brain regions of the mesolimbic system based on the identified relation. Additionally, the one or more stored algorithms are used to modify an interface, for example a feedback interface delivered to the patient according to the detected activation level of the one or more specific brain regions of the mesolimbic system.

According to some exemplary embodiments, the system 602 comprises a patient interface, for example patient interface 620. In some embodiments, the patient interface 620 comprises a display and/or a speaker, configured to deliver a human detectable indication to the patient, for example instructions. Alternatively, the patient interface 620 is configured to deliver at least one neurofeedback signal to the patient 610. In some embodiments, the patient interface comprises an earphone, for example earphone 622. As used herein, an earphone is an interface configured to generate an audio signal directed to the patient, and includes also a headphone. In some embodiments, the patient interface, for example patient interface 620 and/or the earphone 622 is connected to the control unit 604, for example to the control circuitry 614.

According to some exemplary embodiments, the patient interface, for example patient interface 620 and/or the earphones 622 are part of the system 602. Alternatively, the control unit 604 is connectable to a commercially available patient interface.

According to some exemplary embodiments, the control circuitry 614 is configured to determine an activation level of one or more brain regions of the mesolimbic system, for example based on data received from at least one electrode, for example electrodes 606 and 608, or from at least one sensor or detector. In some embodiments, the control circuitry 614 optionally identifies a correlation between the received data and at least one indication stored in the memory, for example an EFP of the one or more brain regions. In some embodiments, the control circuitry 614 signals the patient interface, for example patient interface 620 and/or earphones 622 to generate at least one feedback signal to the patient 610. In some embodiments, the feedback is generated according to at least one of activity of the one or more brain regions, an activity state of the one or more brain regions and/or according to an ability of the patient to modulate the activity of the one or more brain regions. In some embodiments, the control circuitry 614 is configured to modify or to determine how to modify the delivered feedback, according to the at least one of activity of the one or more brain regions, an activity state of the one or more brain regions and/or according to an ability of the patient to modulate the activity of the one or more brain regions.

According to some exemplary embodiments, the neurofeedback system 602 comprises a mobile device, for example a cellular device, which includes at least part of the control unit 604. In some embodiments, the patient interface is an interface of the mobile device, for example a display and/or a speaker of the mobile device. Alternatively or additionally, the patient interface is an interface connectable to the mobile device. In some embodiments, the mobile device is connectable to one or more external electrodes, for example to external EEG electrodes.

### Neurofeedback Proof of Concept Validation Study

Neurofeedback is a training approach in which people learn to regulate their brain activity by using a feedback that reflects their brain activity. An effective utilization of this approach requires that the represented brain activity will be measured with high specificity, yet in an accessible manner, enabling repeated training sessions. To address these issues a Brain Computer Music interface approach was developed. The interface utilizes the fMRI-inspired electroencephalography (EEG) model of mesolimbic activity, centered on the ventral striatum, the VS-EFP, for example as in FIGS. 1a and 1b, and is interfaced with pleasurable self-selected music.

To improve accessibility to the mesolimbic system and reward processes, we developed a feedback interface that is based on pleasurable music. The basic principle behind the musical interface is that during training, participants are presented with their self-selected music, which becomes more or less distorted so as to reliably alter its reward value in real-time. The level of distortion proportionally reflects participants' momentary success in increasing the VS-EFP signal relative to baseline, and is introduced according to a pre-established acoustic filtering procedure. The interface is based on a known capacity of music to induce pleasure in a personalized way, and the generation of dopaminergic responses in the reward circuit, particularly the in the VS. As such, music can serve both as an information-bearing feedback signal and optionally at the same time serve as a robust triggering input to this reward-related circuit.

In a proof of concept study, twenty participants were randomly assigned in a double blind fashions to either test or control NF-groups, where in the test group participants received feedback driven by their own VS-EFP (N=10), and in the control group participants received a sham feedback driven by the VS-EFP of a paired member of the test-group, respectively (N=10). The participants underwent six NF training sessions over the course of 2 to 4 weeks, during which their success in regulating their VS-EFP signal was examined.

To test for learning generalization, participants also underwent a ‘transfer cycle’ where they were requested to volitionally regulate their brain activity with no music nor feedback provided. To further examine (VS) target engagement associated with this procedure, participants also underwent a transfer cycle during an fMRI scan before and after training. To assess the effects of VS-EFP-NF learning on behavioral (and neural) indices of mesolimbic function, participants also completed before and after the training period several tasks that were shown to involve mesolimbic function and to co-vary among individuals with levels of anhedonia; effort expenditure for reward task; Probabilistic selection task, pleasurable music listening task inside the fMRI. Finally, to further assess how individual differences in experienced positive affect and levels of anhedonia are associated with regulation success, participants further completed the PANAS and SHAPS questionnaires, respectively.

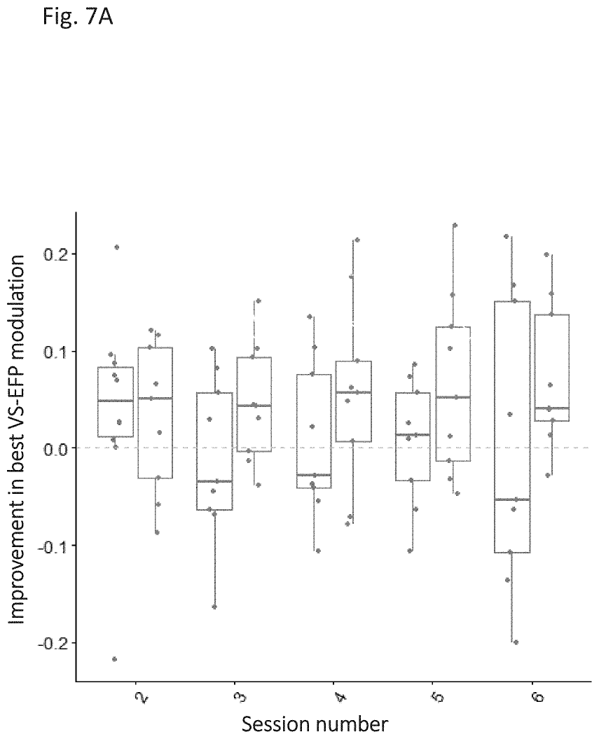

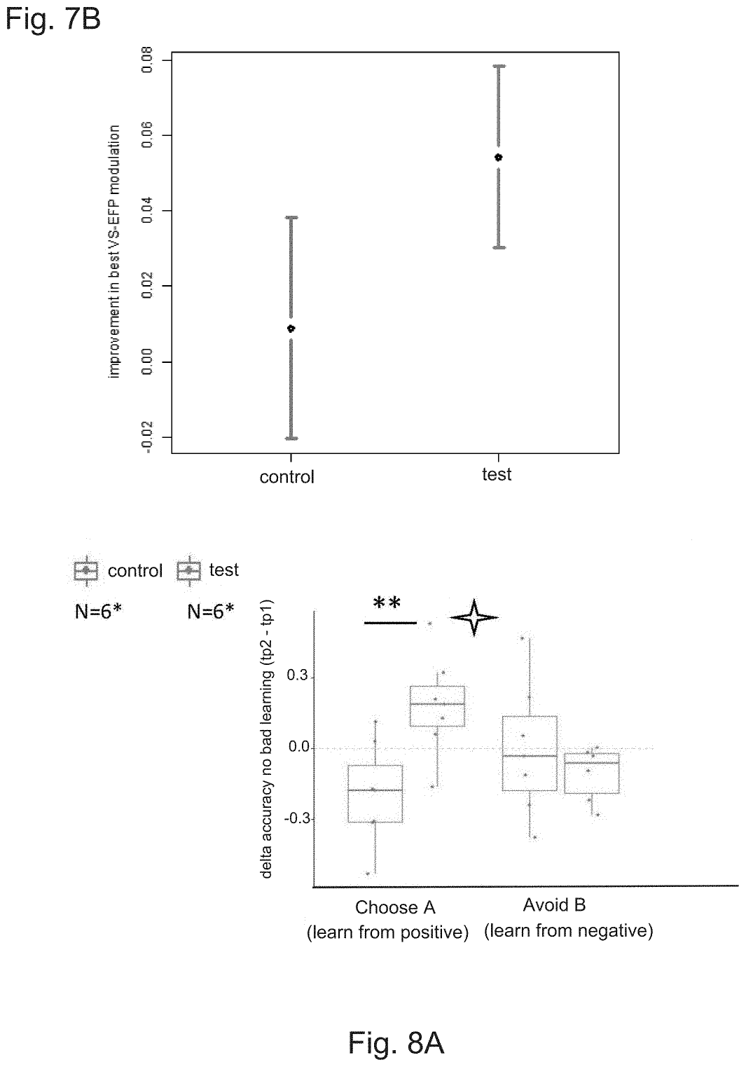

FIG. 7A shows an improvement in performance with respect to the first session calculated in each of the subsequent sessions as the difference between the maximal NF-success (max[Δ, regulate−baseline]) in each session relatively to the maximal performance in the first session. The results show a significant improvement in performance among the test group, but not the control group, starting from session 3. FIG. 7B shows neurofeedback performance in improvement of maximal VS-EFP modulation relative to the first session in the control and test groups, per session.

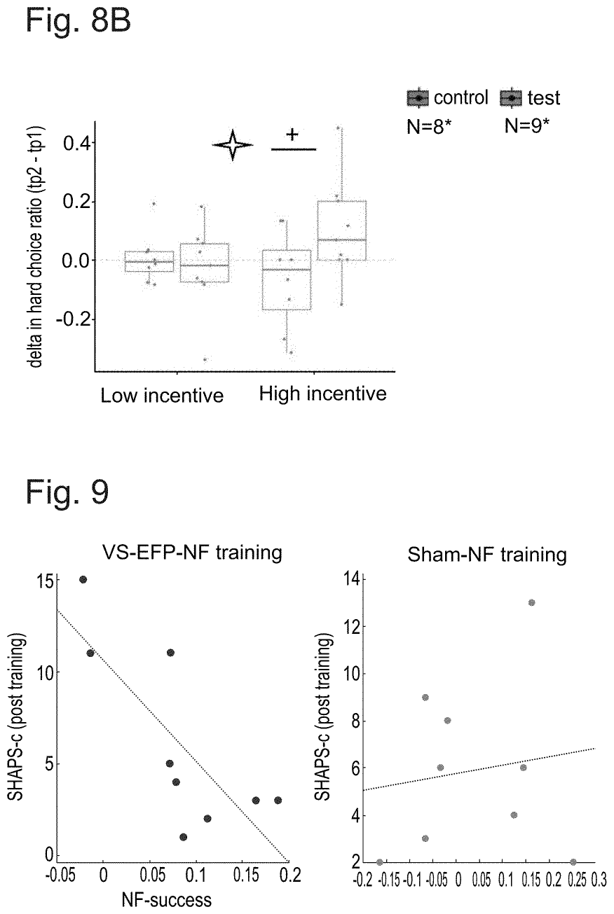

We wished to test if the training had any impact on reward-based processing. We first tested the effects of training on reinforcement learning, by looking at the responses to probabilistic selection task—a task of probabilistic learning which allows disentangling between patterns of learning from rewards or avoiding punishment by assessing the accuracy in selecting an often rewarded symbol (A, rewarded 80% of the trials) or in avoiding a seldom rewarded symbol (B, rewarded only 20% of the trials). Here we examined the difference in such accuracy after-relative to before training. Comparing the improvement in accuracy for both learning from rewards or avoiding punishment, as shown in FIG. 8A revealed that, the test group has showed an improvement in learning from rewards (choose A), which was greater than that of the control group.

We next turn to examine if training also had effect on effort based decision making, which was assessed, for example using the EEfrt task. This task assesses willingness to perform a hard task vs. an easy-motor task for gaining rewards of various magnitudes with varying probabilities of gaining. Effort was quantified as the percentage of choosing the hard task for the chance of earning low- or high monetary gain (>3.5$). Here we examined the difference in such effortful decision relative to before training.

As shown in FIG. 8B, group (VS-EFP-NF vs. sham control) by gain magnitude (high- vs low-) interaction (F(1,15)=4.883, p=0.043) revealed that VS-EFP-NF led to a greater improvement in the willingness to expand effort for high but not low monetary gain than sham-NF (one-tailed post-hoc comparisons: test vs control, high incentive: t(1,14.9)=2.02).

FIG. 9 shows a correlation between VS-EFP neurofeedback performance in the last session and measure of anhedonia gathered following neurofeedback training, for example using the SHAPS questionnaire. The results show a correlation between NF-training success in the last session and measures of Anhedonia in the test group, compared to the control group (Sham-NF training). In the test group, an increase in NF-success, which was indexed as the average VS-EFP power across cycles in the last session is correlated with reduced measures of anhedonia (rₛₚₑₐᵣₘₐₙ=−0.81, p=0.01). No such correlation between NF-performance and reported anhedonia following was evident in the control group (rₛₚₑₐᵣₘₐₙ=−0.19, p=0.62).

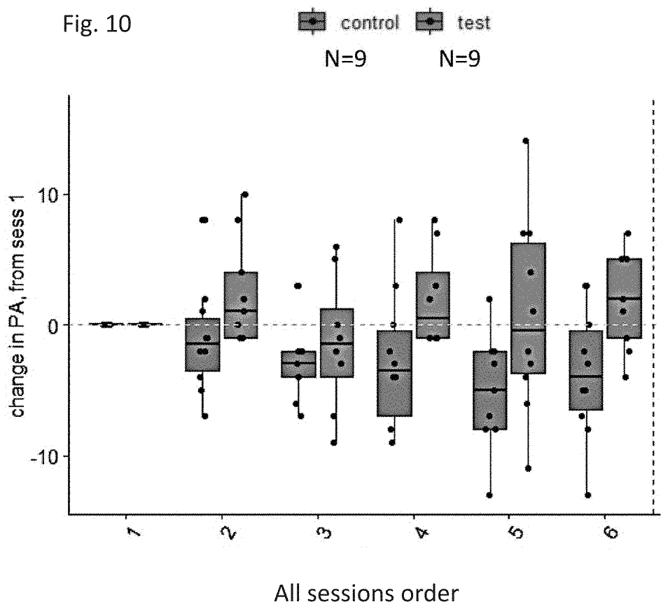

FIG. 10 shows a change in reported positive affect (PA) relative to the first session of NF training. The positive affect was assessed by computing the PA scale from the entries in the PANAS questionnaire, which was administered prior to each meeting. To evaluate if training affected positive affect of participants during training, we assessed the change in reported positive affect at the start of each training session relative to reported PA at the start of the first training session. Analysis shown that the VS-EFP-NF was associated with a relatively higher change in PA relative to sham-NF training across all subsequent sessions (main effect for group, (F(1,99)=16.948, p<0.0001). No such effect was evident for the change in negative affect, as measured using the NA scale of the PANAS questionnaire (F(1,99)=2.1, p=0.15).

It is expected that during the life of a patent maturing from this application many relevant electrical fingerprints will be developed; the scope of the term electrical fingerprint is intended to include all such new technologies a priori. As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A neurofeedback method, using an audio signal having a quality level comprising:
recording electrical signals from at least one brain network of a subject, wherein changes in said recorded electrical signals over time indicate changes in an activity level of said at least one brain network;
providing said audio signal having a perceived degraded quality level based on said recorded electrical signals and according to an activity level of said at least one brain network;
delivering said audio signal to the subject during said recording.

2. A method according to claim 1, comprising degrading said provided audio signal prior to said providing by reducing said quality level of said audio signal.

3. A method according to claim 2, comprising instructing said subject to change said degrading.

4. A method according to claim 2, wherein said audio signal comprises music, and wherein said degrading comprises reducing a perceived quality level of said music.

5. A method according to claim 4, wherein said music is a music selected by the subject as a pleasurable music prior to said degrading.

6. A method according to claim 4, wherein said music is a music affecting mood in said subject.

7. A method according to claim 4, wherein said at least one brain network is a brain network having an activity that is affected by application of said music.

8. A neurofeedback system, comprising:
at least one electrode for recording electrical signals from a subject brain;
memory which stores at least one electrical fingerprint indicating an activity level of at least one deeply located brain region of a mesolimbic system and/or of a reward system;
a user interface configured to generate and deliver a feedback signal to said subject;
a control circuitry configured to;
degrade a stimulus selected to affect an activity of said at least one deeply located brain region of said mesolimbic system and/or of said reward system;
deliver said stimulus to said subject;
receive electrical signals recorded during said delivery of said stimulus by said at least one electrode;
identify a correlation between at least a portion of said recorded electrical signals and said at least one electrical fingerprint;
determine an activation level of said at least one deeply located brain region based on said identified correlation;
modify said degradation of said degraded stimulus according to said determined activation level or changes thereof and
signal said user interface to deliver said modified degraded stimulus to said subject.

9. A method according to claim 1, comprising:
identifying an increase in activation of said at least one specific brain network based on the recorded electrical signals;
changing said perceived degraded quality level of said audio signal by improving said quality level of said audio signal according to said identified increase, and wherein said delivering comprises delivering said audio signal with said improved quality level to the subject during said recording.

10. A method according to claim 1, wherein said delivering comprises delivering continuously said audio signal to the subject during said recording.

11. A method according to claim 1, wherein said brain network comprises a mesolimbic brain network.

12. A method according to claim 1, wherein said recording comprises recording signals of at least one brain region related to said at least one brain network, wherein changes in said recorded electrical signals over time indicate changes in an activity level of said at least one brain region, wherein said providing comprises providing said audio signal having said perceived degraded quality level based on said recorded electrical signals and according to an activity level of said at least one brain region.

13. A method according to claim 12, comprising:
identifying an increase in activation of said at least one specific brain region based on the recorded electrical signals;
changing said perceived degraded quality level of said audio signal by improving said quality level of said audio signal according to said identified increase, and wherein said delivering comprises delivering said audio signal with said improved quality level to the subject during said recording.

14. A method according to claim 12, wherein said at least one brain region comprises a ventral striatum (VS), and wherein said method comprises processing said recorded electrical signals for measuring EEG signals, and using a fingerprint indicating a relation between fMRI-BOLD activity of said ventral striatum and said EEG signals for determining an activity level of said VS or changes thereof.

15. A method according to claim 11, wherein said recording comprises recording said electrical signals from a brain of a subject diagnosed with Anhedonia, and wherein said method comprising:
instructing said subject to perform at least one mental exercise shown to increase the activity level of the mesolimbic brain network during said recording, and
modifying said audio signal having a perceived degraded quality level to a more pleasurable audio signal if activity level of said mesolimbic brain network is increased.

16. A method according to claim 11, wherein said recording comprises recording said electrical signals from a brain of a subject diagnosed with Apathy, and wherein said method comprising:
instructing said subject to perform at least one mental exercise shown to increase the activity level of the mesolimbic brain network during said recording, and
modifying said audio signal having a perceived degraded quality level to a more pleasurable audio signal if activity level of said mesolimbic brain network is increased.

17. A system according to claim 8, wherein said degrade a stimulus by said control circuitry comprises reducing a quality level of said stimulus, and wherein said modify by said control circuitry comprises modify said degradation by improving a quality level of said degraded stimulus if said activity of said at least one deeply located brain region is increased according to results of said determining.

18. A system according to claim 17, wherein said at least one deeply located brain region comprises a ventral striatum (VS), wherein said at least one electrical fingerprint comprises a multi-dimensional model generated by correlating EEG data and fMRI-B OLD activity of the VS, and wherein said control circuitry is configured to measure EEG signals based on said received electrical signals and to identify a correlation between at least a portion of said EEG signals and said at least one electrical fingerprint indicating an activation level of said VS or changes thereof.

19. A system according to claim 17, wherein said stimulus comprises an audio signal, and wherein said control circuitry is configured to degrade said stimulus by degrading a quality level said audio signal and to modify said degradation by improving a quality level of said degraded audio signal if said activity of said at least one deeply located brain region is increased according to results of said determining.

20. A system according to claim 17, wherein said stimulus comprises music, and wherein said control circuitry is configured to degrade said music by degrading a quality level of said music, and to modify said degradation by modifying said music to a more pleasurable music if activity of said at least one deeply located brain region is increased according to results of said determining.
